(19) United States
(12) Patent Application Publication
(10) Pub. No.: US 2005/0130192 A1
(43) Pub. Date: Jun. 16, 2005

(54) Apparatus and method for identifying therapeutic targets using a computer model

(76) Inventors: Thomas S. Paterson, Los Angeles, CA (US); Leif Gustaf Wennerberg, Mountain View, CA (US)

Correspondence Address: FISH & RICHARDSON P.C., PO BOX 1022, MINNEAPOLIS, MN, 55440-1022, US

(21) Appl. No.: 10/938,072

(22) Filed: Sep. 10, 2004

Related U.S. Application Data
(60) Provisional application No. 60/502,333, filed on Sep. 11, 2003.

(51) Int. Cl. C12Q 1/68; G06F 19/00; G01N 33/48; G01N 33/50

(52) U.S. Cl. 435/6; 702/19

(57) ABSTRACT

Apparatus and method for identifying therapeutic targets of a biological system in a disease state are described. In one implementation, a method uses a computer model of the biological system. The method includes identifying a set of functions of a biological constituent of the biological system. The method also includes executing the computer model in the absence of a modification of the set of functions to produce a first output and executing the computer model based on the modification of the set of functions to produce a second output. The method further includes comparing the second output with the first output to identify the biological constituent as a therapeutic target.

### CROSS-REFERENCE TO RELATED APPLICATIONS

This application claims the benefit of U.S. Provisional Application Ser. No. 60/502,333, filed on Sep. 11, 2003, which is hereby incorporated by reference in its entirety.

### COPYRIGHT NOTICE

A portion of the disclosure of the patent document contains material that is subject to copyright protection. The copyright owner has no objection to the facsimile reproduction by anyone of the patent document, as it appears in the Patent and Trademark Office patent file or records, but otherwise reserves all copyright rights whatsoever.

### BACKGROUND

The present invention relates to identifying therapeutic targets.

Drug development can be roughly divided into four stages: discovery, pre-clinical testing, clinical testing, and regulatory approval. As part of the discovery stage, a biological constituent can be identified as a therapeutic target that can be modulated to treat a disease. Currently, the discovery stage provides a significant obstacle to the development of new drugs.

Previous attempts for identifying therapeutic targets sometimes rely on data derived using genomic and proteomic techniques. While genomic and proteomic techniques can correlate changes in gene and protein expression data with a disease, such techniques are often incapable of independently and directly identifying causal relationships. In other words, changes caused by a disease often cannot be distinguished from changes that cause the disease. Moreover, such techniques often cannot predict how changes in gene and protein expression data, which are usually observed in isolated cells or tissue samples, may affect or be affected by a biological system as a whole. Other attempts for identifying therapeutic targets rely on the ability of a researcher to identify causal relationships in the pathophysiology of a disease and to generate a hypothesis regarding biological constituents that can be modulated to treat the disease. Such attempts often require the researcher to acquire and synthesize vast amounts of data and can be tedious and unreliable.

The costs required to successfully bring new drugs to market are enormous and continue to rise. The large numbers of drugs that fail during pre-clinical and clinical testing are a significant contribution to these costs. In particular, about 53 percent of drugs fail during Phase II of clinical trials. A significant proportion of these failures arises from lack of efficacy as a result of pursuing inappropriate therapeutic targets. The quality of a therapeutic target can be affected by unexpected system-wide effects associated with a complex network of biological processes that underlie human physiology. For example, biological redundancies and regulatory feedback control mechanisms can react to molecular interventions from drugs in unexpected ways and can contribute to the ultimate failure of the drugs during pre-clinical and clinical testing.

Conventionally, computer modeling techniques can be used in the drug development process. Computer models can be defined as, for example, described in the following references: Paterson et al., U.S. Pat. No. 6,078,739; Paterson et al., U.S. Pat. No. 6,069,629; Paterson et al., U.S. Pat. No. 6,051,029; Thalhammer-Reyero, U.S. Pat. No. 5,930,154; McAdams et al., U.S. Pat. No. 5,914,891; Fink et al., U.S. Pat. No. 5,808,918; Fink et al., U.S. Pat. No. 5,657,255; Paterson et al., PCT Publication No. WO 99/27443; Paterson et al., PCT Publication No. WO 00/63793; Winslow et al., PCT Publication No. WO 00/65523; and Defranoux et al., PCT Publication No. WO 02/097706; the disclosures of which are incorporated herein by reference in their entirety.

Computer models of particular biological systems are described in the following co-owned and co-pending patent applications: Kelly et al., entitled “Method and Apparatus for Computer Modeling of an Adaptive Immune Response,” U.S. Application Serial No. 10/186,938, filed on Jun. 28, 2002 (U.S. Application Publication No. 20030104475, published on Jun. 5, 2003); Defranoux et al., entitled “Method and Apparatus for Computer Modeling a Joint,” U.S. application Ser. No. 10/154,123, filed on May 22, 2002 (U.S. Application Publication No. 20030078759, published on Apr. 24, 2003); and Brazhnik et al., entitled “Method and Apparatus for Computer Modeling Diabetes,” U.S. application Ser. No. 10/040,373, filed on Jan. 9, 2002 (U.S. Application Publication No. 20030058245, published on Mar. 27, 2003), the disclosures of which are incorporated herein by reference in their entirety.

Commercially available computer models of biological systems are available including Entelos® Asthma PhysioLab® systems, Entelos® Metabolism PhysioLab® systems, and Entelos® Adipocyte CytoLab® systems.

Computer models can be validated. Examples of techniques for validation are described in the co-pending and co-owned patent application to Paterson, entitled “Apparatus and Method for Validating a Computer Model”, U.S. application Ser. No. 10/151,581, filed on May 16, 2002 (U.S. Application Publication No. 20020193979, published on Dec. 19, 2002), the disclosure of which is incorporated herein by reference in its entirety.

### SUMMARY

In general, in one aspect, the invention features a method of identifying a therapeutic target of a biological system. The method includes receiving a computer model of a biological system, the model including a plurality of model processes representing a plurality of biological processes and operable to model one or more clinical outcomes associated with a particular disease state. The method includes receiving user input identifying one or more biological processes of the plurality of biological processes, the one or more biological processes being identified as being associated with the one or more clinical outcomes. The method further includes modifying, from user input, one or more parameters in the computer model for one or more model processes corresponding to the one or more identified biological processes and running the computer model using the modified parameters for the one or more model processes to produce output values modeling one or more clinical outcomes. The method further includes identifying one or more modified model processes as a potential therapeutic target.

Advantageous implementations of the invention include one or more of the following features. Identifying one or more model processes can include providing filter information related to the output values. The method of identifying a therapeutic target can further include providing the output values as a graphical output for the one or more clinical outcomes. The method of identifying a therapeutic target can further include examining each potential therapeutic target for use as a therapeutic target for treating the disease state, including. Examining each potential therapeutic target can include receiving a user identified biological constituent operable to modify a function of a biological process identified as a potential therapeutic target, receiving user input incorporating a model constituent representing the biological constituent into the computer model of the biological system, modeling the effect of the model constituent on the one or more model processes associated with the one or more clinical outcomes, and modeling the effect of the one or more model processes affected by the model constituent on the one or more clinical outcomes. The method can include validating the effect of the biological constituent on the one or more clinical outcomes using biological assays.

In general, in one aspect, the invention features a method of identifying a therapeutic target of a biological system. The method includes receiving a user identification of a biological constituent selected as a potential therapeutic target for treating a particular disease state. The method includes receiving a computer model of a biological system including a plurality of functions associated and operable to model one or more clinical outcomes associated with a particular disease state. The method includes receiving a user input modifying one or more functions of the plurality of functions affected by the biological constituent. The method includes using the computer model to perform a sensitivity analysis on the one or more functions affected by the biological constituent to identify a set of functions of the one or more functions associated with one or more clinical outcomes and modeling the effect of the identified set of functions affected by the biological constituent on the one or more clinical outcomes.

In general, in one aspect, the invention features a method of identifying a therapeutic target of a biological system in a disease state. The method includes identifying a set of functions of a biological constituent of the biological system. The method also includes executing a computer model in the absence of a modification of the set of functions to produce a first output and executing the computer model based on the modification of the set of functions to produce a second output. The method further includes comparing the second output with the first output to identify the biological constituent as a therapeutic target.

In general, in another aspect, the invention features a method of identifying a therapeutic target of a biological system in a disease state. The method includes executing a computer model to identify a set of biological processes that contribute to the occurrence of the disease state. The set of biological processes is a subset of the various biological processes. The method also includes identifying a biological constituent associated with the set of biological processes and identifying a set of functions of the biological constituent. Each function of the set of functions is associated with at least one biological process of the various biological processes. The method also includes executing the computer model in the absence of a modification of the set of functions to produce a first output and executing the computer model based on the modification of the set of functions to produce a second output. The method further includes comparing the second output with the first output to identify the biological constituent as a therapeutic target.

In a further innovative aspect, the invention relates to a computer-readable medium. In one embodiment, the computer-readable medium includes code to define a computer model of a biological system in a disease state. The computer model represents a set of functions of a biological constituent of the biological system. The computer-readable medium also includes code to define a virtual stimulus. The virtual stimulus represents a modification of the set of functions. The computer-readable medium further includes code to execute the computer model in the absence of the virtual stimulus to produce a first output and code to execute the computer model based on the virtual stimulus to produce a second output.

The invention can be implemented to realize one or more of the following advantages. Potential therapeutic targets can be identified using computer modeling techniques. The use of the techniques for identifying therapeutic targets assists in developing drugs to treat various diseases, such as, for example, asthma, diabetes, obesity, and rheumatoid arthritis. The computer model are used to identify biological processes associated with clinical outcomes for a particular disease state. A biological constituent is identified as potentially effecting functions associated with the identified biological processes. A set of biological processes or functions of a biological constituent is identified and tested using a computer model of the biological system. A computer model is used to determine whether any of the identified biological processes or functions affects clinical outcomes for a particular disease state. The computer model can prioritize experimental work to enhance the probability of identifying successful therapeutic targets, and the probability of stopping further work on unsuccessful targets.

A sensitivity analysis is performed to determine the importance of a particular biological process or a particular function in the context of a disease state. Sensitivity analysis allows for prioritization of biological processes that are associated with the disease state. A computer model is used to model the effects of a particular biological constituent on one or more functions associated with a diseased state. The computer model can further model the combined effects of a biological constituent on the clinical outcome of a disease state.

The details of one or more embodiments of the invention are set forth in the accompanying drawings and the description below. Other features and advantages of the invention will become apparent from the description, the drawings, and the claims.

### BRIEF DESCRIPTION OF THE DRAWINGS

FIG. 1 shows a flow chart of a method for identifying therapeutic targets.

FIG. 2 shows an example of a diagram of a portion of a computer model representing cartilage matrix metabolism in a joint.

FIGS. 3A and 3B show bar charts for two different virtual patients that can be defined to represent different human patient types.

FIG. 4 and FIG. 5 show outputs based on sensitivity analysis of various biological processes associated with a joint in a disease state.

FIG. 6 shows a flow chart of a method for examining a potential therapeutic target FIGS. 7 and 8 show outputs based on sensitivity analysis of biological processes or functions associated with biological constituent CD99.

FIGS. 9 and 10 show additional outputs based on sensitivity analysis of biological processes or functions associated with biological constituent CD99.

FIG. 11 shows outputs based on combined effects of CD99.

FIG. 12 shows a flow chart of a method for identifying a therapeutic target.

FIG. 13 shows outputs based on sensitivity analysis of various potential functions affected by biological constituent p38.

FIG. 14 shows example outputs based on effects of p38.

FIG. 15 shows a flow chart for identifying a therapeutic target.

FIG. 16 shows a system block diagram of a computer system.

Like reference numbers and designations in the various drawings indicate like elements.

### DETAILED DESCRIPTION

### Definitions

The following definitions apply to some of the elements described with regard to some implementations of the invention. These definitions may likewise be expanded upon herein.

The term “biological constituent” refers to a portion of a biological system. A biological system can include, for example, an individual cell, a collection of cells such as a cell culture, an organ, a tissue, a multi-cellular organism such as an individual human patient, a subset of cells of a multi-cellular organism, or a population of multi-cellular organisms such as a group of human patients or the general human population as a whole. A biological system can also include, for example, a multi-tissue system such as the nervous system, immune system, or cardio-vascular system. A biological constituent that is part of a biological system can include, for example, an extra-cellular constituent, a cellular constituent, an intra-cellular constituent, or a combination of them. Examples of biological constituents include DNA; RNA; proteins; enzymes; hormones; cells; organs; tissues; portions of cells, tissues, or organs; subcellular organelles such as mitochondria, nuclei, Golgi complexes, lysosomes, endoplasmic reticula, and ribosomes; chemically reactive molecules such as H⁺; superoxides; ATP; citric acid; protein albumin; and combinations of them.

The term “function” with reference to a biological constituent refers to an interaction of the biological constituent with one or more additional biological constituents. Each biological constituent of a biological system can interact according to some biological mechanism with one or more additional biological constituents of the biological system. A biological mechanism by which biological constituents interact with one another can be known or unknown. A biological mechanism can involve, for example, a biological system's synthetic, regulatory, homeostatic, or control networks. For example, an interaction of one biological constituent with another can include, for example, a synthetic transformation of one biological constituent into the other, a direct physical interaction of the biological constituents, an indirect interaction of the biological constituents mediated through intermediate biological events, or some other mechanism. In some instances, an interaction of one biological constituent with another can include, for example, a regulatory modulation of one biological constituent by another, such as an inhibition or stimulation of a production rate, a level, or an activity of one biological constituent by another.

The term “biological state” refers to a condition associated with a biological system. In some instances, a biological state refers to a condition associated with the occurrence of a set of biological processes of a biological system. Each biological process of a biological system can interact according to some biological mechanism with one or more additional biological processes of the biological system. As the biological processes change relative to each other, a biological state typically also changes. A biological state typically depends on various biological mechanisms by which biological processes interact with one another. A biological state can include, for example, a condition of a nutrient or hormone concentration in plasma, interstitial fluid, intracellular fluid, or cerebrospinal fluid. For example, biological states associated with hypoglycemia and hypoinsulinemia are characterized by conditions of low blood sugar and low blood insulin, respectively. These conditions can be imposed experimentally or can be inherently present in a particular biological system. As another example, a biological state of a neuron can include, for example, a condition in which the neuron is at rest, a condition in which the neuron is firing an action potential, a condition in which the neuron is releasing a neurotransmitter, or a combination of them. As a further example, biological states of a collection of plasma nutrients can include a condition in which a person awakens from an overnight fast, a condition just after a meal, and a condition between meals. As another example, biological state of a rheumatic joint can include significant cartilage degradation and hyperplasia of inflammatory cells.

A biological state can include a “disease state,” which refers to an abnormal or harmful condition associated with a biological system. A disease state is typically associated with an abnormal or harmful effect of a disease in a biological system. In some instances, a disease state refers to a condition associated with the occurrence of a set of biological processes of a biological system, where the set of biological processes play a role in an abnormal or harmful effect of a disease in the biological system. A disease state can be observed in, for example, a cell, an organ, a tissue, a multi-cellular organism, or a population of multi-cellular organisms. Examples of disease states include conditions associated with asthma, diabetes, obesity, and rheumatoid arthritis.

The term “biological process” refers to an interaction or a set of interactions between biological constituents of a biological system. In some instances, a biological process can refer to a set of biological constituents drawn from some aspect of a biological system together with a network of interactions between the biological constituents. Biological processes can include, for example, biochemical or molecular pathways. Biological processes can also include, for example, pathways that occur within or in contact with an environment of a cell, organ, tissue, or multi-cellular organism. Examples of biological processes include biochemical pathways in which molecules are broken down to provide cellular energy, biochemical pathways in which molecules are built up to provide cellular structure or energy stores, biochemical pathways in which proteins or nucleic acids are synthesized or activated, and biochemical pathways in which protein or nucleic acid precursors are synthesized. Biological constituents of such biochemical pathways include, for example, enzymes, synthetic intermediates, substrate precursors, and intermediate species.

Biological processes can also include, for example, signaling and control pathways. Biological constituents of such pathways include, for example, primary or intermediate signaling molecules as well as proteins participating in signaling or control cascades that usually characterize these pathways. For signaling pathways, binding of a signaling molecule to a receptor can directly influence the amount of intermediate signaling molecules and can indirectly influence the degree of phosphorylation (or other modification) of pathway proteins. Binding of signaling molecules can influence activities of cellular proteins by, for example, affecting the transcriptional behavior of a cell. These cellular proteins are often important effectors of cellular events initiated by a signal. Control pathways, such as those controlling the timing and occurrence of cell cycles, share some similarities with signaling pathways. Here, multiple and often ongoing cellular events are temporally coordinated, often with feedback control, to achieve an outcome, such as, for example, cell division with chromosome segregation. This temporal coordination is a consequence of the functioning of control pathways, which are often mediated by mutual influences of proteins on each other's degree of modification or activation (e.g., phosphorylation). Other control pathways can include pathways that can seek to maintain optimal levels of cellular metabolites in the face of a changing environment.

Biological processes can be hierarchical, non-hierarchical, or a combination of hierarchical and non-hierarchical. A hierarchical process is one in which biological constituents can be arranged into a hierarchy of levels, such that biological constituents belonging to a particular level can interact with biological constituents belonging to other levels. A hierarchical process generally originates from biological constituents belonging to the lowest levels. A non-hierarchical process is one in which a biological constituent in the process can interact with another biological constituent that is further upstream or downstream. A non-hierarchical process often has one or more feedback loops. A feedback loop in a biological process refers to a subset of biological constituents of the biological process, where each biological constituent of the feedback loop can interact with other biological constituents of the feedback loop.

The term “patient” refers to a biological system to which a therapy can be administered. A patient can refer to a human patient or a non-human patient. In some instances, a patient can have a disease, such as, for example, rheumatoid arthritis. Patients having a disease can include, for example, patients that have been diagnosed with the disease, patients that exhibit a set of symptoms associated with the disease, and patients that are progressing towards or are at risk of developing the disease.

The term “therapy” refers to a type of stimulus or perturbation that can be applied to a biological system. In some instances, a therapy can affect a biological state of a biological system by known or unknown biological mechanisms. Therapies that can be applied to a biological system can include, for example, drugs, environmental changes, or combinations of them.

The term “drug” refers to a compound of any degree of complexity that can affect a biological state, whether by known or unknown biological mechanisms, and whether or not used therapeutically. In some instances, a drug exerts its effects by interacting with a biological constituent, which can be referred to as a therapeutic target of the drug. A drug that stimulates a function of a therapeutic target can be referred to as an “activating drug” or an “agonist,” while a drug that inhibits a function of a therapeutic target can be referred to as an “inhibiting drug” or an “antagonist.” An effect of a drug can be a consequence of, for example, drug-mediated changes in the rate of transcription or degradation of one or more species of RNA, drug-mediated changes in the rate or extent of translational or post-translational processing of one or more polypeptides, drug-mediated changes in the rate or extent of degradation of one or more proteins, drug-mediated inhibition or stimulation of action or activity of one or more proteins, and so forth. Examples of drugs include typical small molecules of research or therapeutic interest; naturally-occurring factors such as endocrine, paracrine, or autocrine factors or factors interacting with cell receptors of any type; intracellular factors such as elements of intracellular signaling pathways; factors isolated from other natural sources; pesticides; herbicides; and insecticides. Drugs can also include, for example, agents used in gene therapy like DNA and RNA. Also, antibodies, viruses, bacteria, and bioactive agents produced by bacteria and viruses (e.g., toxins) can be considered as drugs. For certain applications, a drug can include a composition including a set of drugs or a composition including a set of drugs and a set of excipients.

### Overview

A number of different biological processes or functions can affect the behavior of a particular biological system. Some biological processes or functions have a greater effect on the biological system than others with respect to a particular biological condition such as a particular disease state (e.g., rheumatoid arthritis, diabetes, obesity, and asthma). Identifying the effects of different biological processes or functions can lead to development of different treatments for a particular disease state. Computer modeling can be used to help identify potential targets for treating a particular disease state.

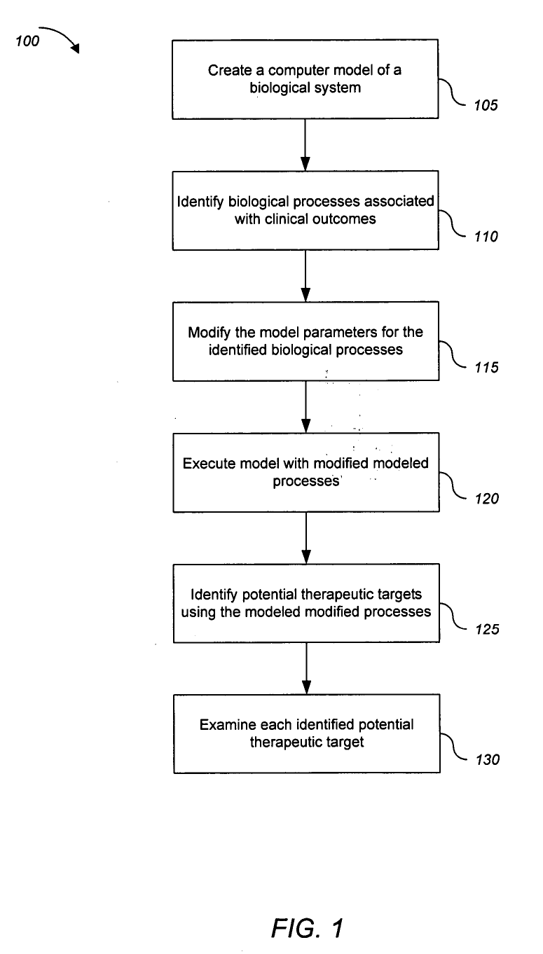

FIG. 1 shows a method 100 for identifying therapeutic targets. The method 100 begins with the creation of a computer model for a biological system that includes a particular set of biological process (step 105). The computer model provides a top down model of behaviors for a particular disease state. The behaviors indicative of a particular disease state includes modeled biological processes and functions associated with the disease state. The model allows identification of one or more biological processes for analysis. The identified biological processes are associated with particular clinical outcomes for a disease state (step 110). During the analysis, the computer modeler modifies parameters of each modeled biological process to provide a range of output values (step 115). The effects of each biological process are modeled over the range of values (step 120). A user can identify biological processes as potential therapeutic targets using the output values (step 125). The identified potential therapeutic targets are then each examined for use as a therapeutic target (step 130). Examining each potential therapeutic target includes identifying a biological constituent capable of modifying the therapeutic target. Method 100 can be used to identify potential targets relevant to rheumatoid arthritis, asthma, diabetes, or obesity.

### Modeling a Biological System (step 105)

The computer model created in step 105 is used to model one or more biological processes or functions. The computer model is built using a “top-down” approach that begins by defining a general set of behaviors indicative of the disease. The behaviors are then used as constraints on the system and a set of nested subsystems are developed to define the next level of underlying detail. For example, given a behavior such as cartilage degradation in rheumatoid arthritis, the specific mechanisms inducing the behavior are each be modeled in turn, yielding a set of subsystems, which can themselves be deconstructed and modeled in detail. The control and context of these subsystems is, therefore, already defined by the behaviors that characterize the dynamics of the system as a whole. The deconstruction process continues modeling more and more biology, from the top down, until there is enough detail to replicate a given biological behavior. Specifically, the model is capable of modeling biological processes that can be manipulated by a drug or other therapeutic agent.

In one implementation, a computer model is created that implements a mathematical model representing a set of biological processes or functions associated with a biological system defined by a set of mathematical relations. For example, the computer model represents a first biological process using a first mathematical relation and a second biological process using a second mathematical relation. A mathematical relation typically includes one or more variables. The computer model simulates the behavior (e.g., time evolution) of the one or more variables. More particularly, mathematical relations of the computer model define interactions among variables, where the variables represent levels or activities of various biological constituents of the biological system as well as levels or activities of combinations or aggregate representations of the various biological constituents. Additionally, variables also represent stimuli that can be applied to the biological system.

A computer model typically includes a set of parameters that affect the behavior of the variables included in the computer model. For example, the parameters represent initial values of variables, half-lives of variables, rate constants, conversion ratios, and exponents. These variables typically admit a range of values, due to variability in experimental systems. Specific values are chosen to give constituent and system behaviors consistent with known constraints. Thus, the behavior of a variable in the computer model changes over time. The computer model includes the set of parameters in the mathematical relations. In one implementation, the parameters are used to represent intrinsic characteristics (e.g., genetic factors) as well as external characteristics (e.g., environmental factors) for a biological system.

Mathematical constructs implemented in a computer model can include, for example, ordinary differential equations, partial differential equations, stochastic differential equations, differential algebraic equations, difference equations, cellular automata, coupled maps, equations of networks of Boolean, fuzzy logical networks, or a combination of them.

Executing the computer model produces a set of outputs for a biological system represented by the computer model. The set of outputs represent one or more biological states of the biological system and includes values or other indicia associated with variables and parameters at a particular time and for a particular execution scenario. For example, a biological state is represented by values at a particular time. The behavior of the variables is simulated by, for example, numerical or analytical integration of one or more mathematical relations produce values for the variables at various times and hence the evolution of the biological state over time.

In one implementation, the created computer model can represent a normal state as well as a disease state of a biological system. For example, the computer model includes parameters that are altered to simulate a disease state or a progression towards the disease state. By selecting and altering one or more parameters, a user modifies a normal state and induces a disease state of interest. In one implementation, selecting or altering one or more parameters is performed automatically.

The created computer model represents biological processes at one hierarchical level and then evaluates the effect of the biological processes on biological processes at a different hierarchical level. Thus, the created computer model provides a multi-variable view of a biological system. The created computer model also provides cross-disciplinary observations through synthesis of information from two or more disciplines into a single computer model or through linking two computer models that represent different disciplines.

In another implementation, the computer model is hierarchical and reflects a particular biological system and anatomical factors relevant to issues to be explored by the computer model. The level of detail at which a hierarchy starts and the level of detail at which the hierarchy ends are often dictated by a particular intended use of the computer model. For example, biological constituents being evaluated often operate at a subcellular level, therefore, the subcellular level can occupy the lowest level of the hierarchy. The subcellular level includes, for example, biological constituents such as DNA, mRNA, proteins, chemically reactive molecules, and subcellular organelles. Because an individual biological system is a common entity of interest with respect to the ultimate effect of the biological constituents, the individual biological system (e.g., represented in the form of clinical outcomes) is at the highest level of the hierarchy.

In one implementation, the computer model is configured to allow visual representation of mathematical relations as well as interrelationships between variables, parameters, and biological processes. This visual representation includes multiple modules or functional areas that, when grouped together, represent a large complex model of a biological system.

Modeling a Joint

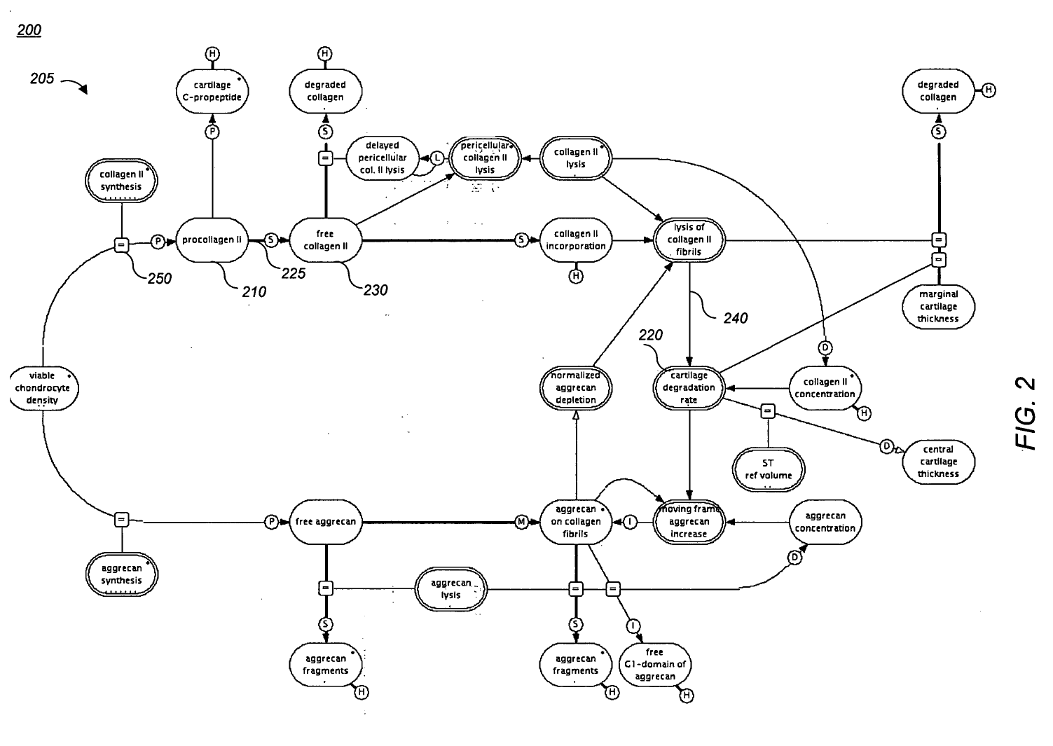

In one implementation, a computer model is created in step 105 to represent part of a joint, for example, a joint representing a diseased state such as rheumatoid arthritis. FIG. 2 shows a diagram of a portion 205 of a computer model 200 representing some of the biological processes for the joint. In particular, FIG. 2 shows cartilage matrix metabolism in the joint. Cartilage matrix metabolism effects different joint disease states including rheumatoid arthritis. The portion 205 includes biological processes related to cartilage degradation rate, which is a clinical outcome for rheumatoid arthritis.

The portion of computer model 200 shows a structural representation of the computer model including a number of different nodes. The nodes represent variables included in computer model 200. For example, the nodes represent parameters and mathematical relations included in computer model 200. Examples of the types of nodes are discussed below.

State nodes (e.g., state node 210), are represented in the computer model 200 as single-border ovals. The state nodes represent variables having values that can be determined by cumulative effects of inputs over time. In one implementation, values of state nodes are determined using differential equations. Parameters associated with each state node include an initial value (Sₒ) and a status (e.g., value of the state node can be computed, held constant, or varied in accordance with specified criteria). A state node can be associated with a half-life and can be labeled with a half-life “H” symbol. An example of a state node is node 210 which represents procollagen.

Function nodes (e.g., function node 220), are represented in the computer model 200 as double-border ovals. The function nodes represent variables having values that, at a particular point in time, are determined by inputs at that same point in time. Values of function nodes are determined using mathematical functions of inputs. Parameters associated with a function node include an initial value and a status (e.g., value of the function node can be computed, held constant, or varied in accordance with specified output values corresponding to given inputs) as well as other parameters necessary to evaluate the functions. An example of a function node is node 220 which represents the cartilage degradation rate.

The nodes are linked together within computer model 200 by lines and arrows. The arrows represent relationships between different nodes. Conversion arrows (e.g., arrow 225), are represented in computer model 200 as thick arrows. Conversion arrows represent a conversion of one or more variables represented by connected nodes. Each conversion arrow includes a label that indicates a type of conversion for the one or more variables. For example, a label of a conversion arrow with a “M” indicate a movement while a label of a “S” indicate a change of state of one or more variables. The computer model 200 also includes argument arrows 240. The argument arrows specify which nodes are inputs for the function nodes (e.g., function node 220).

The computer model 200 also includes modifiers (e.g., modifier 250). Modifiers indicate the effects that particular nodes have on the arrows to which they are connected.

Their effect is to allow time varying biological states to affect the rates of change of state nodes. The types of effects are qualitatively indicated by symbols in the boxes shown in FIG. 2. For example, a node can allow “A”, block “B”, regulate “=”, inhibit “−”, or stimulate a relationship represented by an arrow.

The computer model 200, therefore, illustrates the interactions between biological constituents associated with cartilage matrix metabolism. For example, node 210 represents procollagen. A conversion arrow 225 connects node 210 with node 230 representing free collagen. The conversion arrow 225 represents the conversion from procollagen to free collagen as part of the cartilage matrix metabolism process.

In one implementation, the computer model 200 includes one or more configurations. Various configurations of the computer model 200 are associated with different representations of a biological system. In particular, various configurations of the computer model 200 represent, for example, different variations of the biological system having different intrinsic characteristics, different external characteristics, or both. An observable condition (e.g., an outward manifestation) of a biological system is referred to as its phenotype, while underlying conditions of the biological system that give rise to the phenotype can be based on genetic factors, environmental factors, or both. Phenotypes of a biological system are defined with varying degrees of specificity. In some instances, a phenotype includes an outward manifestation associated with a disease state. A particular phenotype typically is reproduced by different underlying conditions (e.g., different combinations of genetic and environmental factors). For example, two human patients may appear to be similarly arthritic, but one can be arthritic because of genetic susceptibility, while the other can be arthritic because of diet and lifestyle choices.

Virtual Patients

A configuration of the computer model represents different underlying conditions giving rise to a particular biological system phenotype. Additionally, various configurations of the computer model 200 can represent different phenotypes of the biological system. In one implementation, a particular configuration of the computer model 200 is referred to as a virtual patient. A virtual patient represents a human patient having a phenotype based on a particular combination of underlying conditions. Various virtual patients represent human patients having the same phenotype but based on different underlying conditions. For example, as described above, the phenotype of arthritis has a first underlying set of conditions related to genetic susceptibility and a second underlying set of conditions related to diet and lifestyle choices. In an alternative implementation, various virtual patients are developed to represent human patients having different phenotypes. Different virtual patients respond differently to a specified therapy because of their differing underlying characteristics.

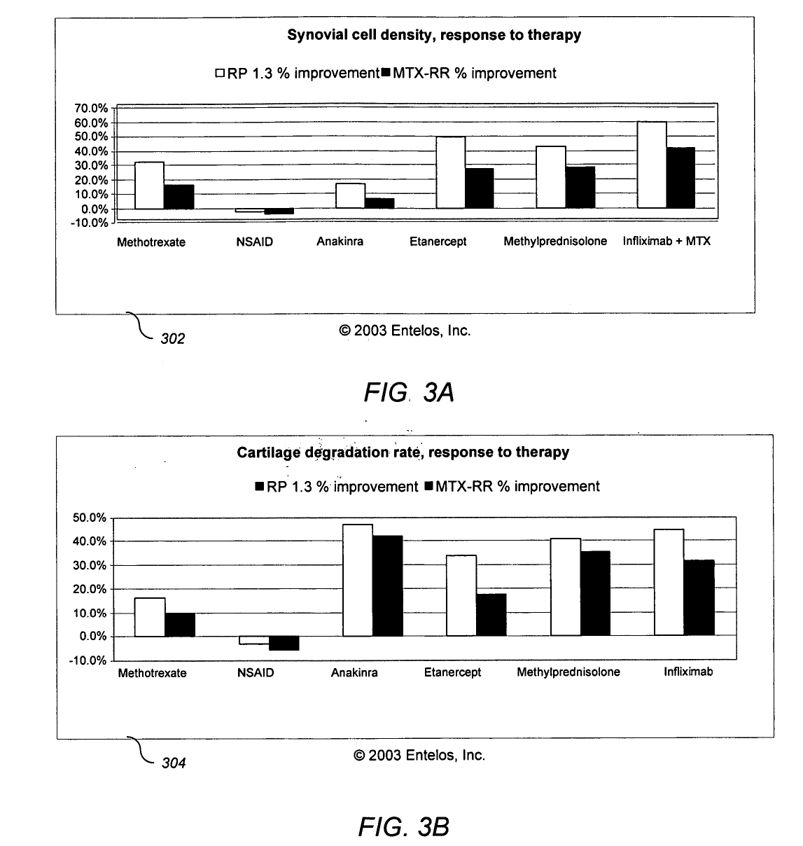

FIGS. 3A and 3B show bar charts, 302 and 304 respectively, for two virtual patients representing different human patients. A first virtual patient (labeled as “RP 1.3”) represents an arthritic human patient that exhibits appropriate responses to common therapies for rheumatoid arthritis, and a second virtual patient (labeled as “MTX-RR”) represents an arthritic human patient that exhibits reduced response to methotrexate, a conventional treatment for arthritis. Each virtual patient is associated with a particular set of values for parameters of the computer model. For example, parameter values associated with IL-4 synthesis, expression of P-selectin, and macrophage apoptosis can be specified to represent the different arthritic human patients (i.e., different virtual patients can have different parameter values for biological processes associated with rheumatoid arthritis). Virtual therapies can be simulated to evaluate the behavior of the virtual patients based on the virtual therapies. The outputs of the virtual therapies are shown for each virtual patient in FIGS. 3A and 3B. In particular, six different virtual therapies for rheumatoid arthritis are shown. FIG. 3A shows outputs of the six therapies for virtual patient RP 1.3 and virtual patent MTX-RR on synovial cell density. FIG. 3B shows outputs of the six therapies for virtual patient RP 1.3 and virtual patent MTX-RR on cartilage degradation rate. Outputs of the virtual therapies are expressed as a percentage improvement in synovial cell density and cartilage degradation rate. Synovial cell density and cartilage degradation rate are clinical outcomes associated with rheumatoid arthritis. A decrease in synovial cell density and cartilage degradation rate can be indicative of effectiveness of a therapy for rheumatoid arthritis.

As shown in FIGS. 3A and 3B, the outputs of the virtual therapies differ between the two virtual patients. Consequently, the effectiveness of a particular therapy can depend upon the characteristics of the particular patient. For example, the effect on synovial cell density in response to methotrexate treatment for a methotrexate resistant patient (e.g., virtual patient MTX-RR) is substantially less then the effect for a non resistant patient (e.g., virtual patient RP 1.3). The computer model examines therapeutic effects for various virtual patients representing different patient types for the same disease.

In one implementation, a configuration of the computer model 200 is associated with a particular set of values for parameters of the computer model 200. Thus, a first configuration is associated with a first set of parameter values, and a second configuration is associated with a second set of parameter values having values of one or more parameters that are distinct from the first set of parameter values. One or more configurations of the computer model are created based on an initial configuration that is associated with initial parameter values. A different configuration is created based on the initial configuration by modifying the initial configuration, for example, by modifying one or more of the initial parameter values. The alternative parameter values are grouped into different sets of parameter values used to define different configurations of the computer model 200. In one implementation, one or more configurations of the computer model are created based on the initial configuration using linked simulation operations as, for example, disclosed in the co-pending and co-owned patent application to Paterson et al., entitled “Method and Apparatus for Conducting Linked Simulation Operations Utilizing A Computer-Based System Model”, U.S. application Ser. No. 09/814,536, filed on Mar. 21, 2001 (U.S. Application Publication No. 20010032068, published on Oct. 18, 2001), the disclosure of which is incorporated herein by reference in its entirety.

In one implementation, various configurations of the computer model 200 represent variations of a biological system that are sufficiently different, such that the effect of such variations on a response of the biological system to a stimulus is evaluated. For example, a set of biological processes represented by the computer model 200 is identified by a user as being associated with a particular disease state, and different configurations represent different modifications of the set of biological processes. A user can identify the set of biological processes using, for example, experimental data, clinical data, knowledge or opinion of persons skilled in the art, outputs of the computer model, and other relevant sources. Once the set of biological processes have been identified, different configurations are created by defining modifications to a set of mathematical relations included in the computer model representing the set of biological processes.

The different behaviors of the different configurations of the computer model 200 are used for predictive analysis. In particular, a set of configurations is used to predict the behavior of different representations of a biological system when subjected to various stimuli. A virtual stimulus simulates a stimulus or perturbation applied to the biological system. The computer model 200 is run based on the virtual stimulus to obtain a set of outputs for the biological system. In one implementation, a virtual stimulus simulates a therapy administered to the biological system. The virtual stimulus is referred to as a virtual therapy. For example, the computer model includes parameters that are altered to simulate the administration of a therapy for rheumatoid arthritis, for example, the administration of methotrexate.

### Identifying Biological Processes Associated with Clinical Outcomes (Step 110)

Referring back to FIG. 1, at step 110, a set of biological processes associated with clinical outcomes for a particular disease state are identified. The biological processes are represented within the created computer model 200 for a particular biological system. In an alternative implementation, a set of biological processes for a particular biological constituent are first identified by a user and then integrated into a computer model. The set of biological processes associated with the disease state typically will include, for example, biological processes affecting (e.g., causing) the disease state, biological processes that are affected by the disease state, or a combination of them.

In one example, the disease state is associated with rheumatoid arthritis. Rheumatoid arthritis is an inflammatory disease characterized by a number of symptoms, including increased synovial cell density, increased cartilage degradation rate, and increased pro-inflammatory cytokine levels (e.g., increased IL-6 levels) in synovial fluid. The symptoms are referred to as clinical outcomes of rheumatoid arthritis. In this example, the set of biological processes includes biological processes that affect rheumatoid arthritis, biological processes that are affected as a result of rheumatoid arthritis, or a combination of them.

The set of biological processes are identified by a user from information available in the art regarding the disease state, or information available in the art regarding biological processes of the biological system. Information typically used to identify the set of biological processes includes experimental data, clinical data, knowledge or opinion of persons skilled in the art, outputs of the computer model, and other relevant sources.

Alternatively, a user identifies the set of biological processes using an execution of the computer model of the biological system. The computer model represents various biological processes of the biological system, and the computer model models the effect of the various biological processes on the disease state. For example, the computer model represents various biological processes of a joint in a disease state as shown, for example, in computer model 200 (FIG. 2). Computer model 200 models various biological processes associated with cartilage matrix metabolism. Computer model 200 models the effect of the different biological processes on the clinical outcomes associated with the disease state (e.g., the effects of different biological processes on rheumatoid arthritis). The outputs of the computer model include values representing levels or activities of biological constituents or any other behavior of the disease state, including effects on the clinical outcomes of the virtual stimuli applied to the modeled biological system.

Using the outputs, a set of biological processes are identified as being associated with the disease state. The user identifies the set of biological processes using the computer modeled outputs. For rheumatoid arthritis, the disease state is represented as outputs associated with, for example, enzyme activities, product formation dynamics, and cellular functions that can indicate one or more biological processes that affect or are affected by the disease state. For example, biological processes associated with rheumatoid arthritis include regulation of macrophage apoptosis, monocyte recruitment rate, T-cell apoptosis rate, T-cell recruitment rate, and T-cell IFNg production.

### Modifying Parameters of the Identified Biological Processes (Step 115)

Referring again to FIG. 1, after one or more biological processes have been identified as producing outputs associated with the clinical outcomes, the parameters of each biological process are modified in step 115 to model, for example, an inhibition or a stimulation of the biological process. The computer model 200 applies the modification of the modeled biological process to identify a degree of connection (e.g., a degree of correlation) between the biological process and the disease state. For example, modifying a modeled biological process is used to identify the impact of the biological process on the disease state. A biological process contributes to the occurrence of the disease state if a modification of the biological process produces or increases the severity of the disease state. In one implementation, modifying a modeled biological process is used to identify the degree of connection between other biological processes and the disease state.

Specifically, modifying one or more mathematical relations representing an identified biological process represents a modification of the biological process. Modifying a mathematical relation includes, for example, a parametric change (e.g., altering or specifying one or more parameter values associated with the mathematical relation), altering or specifying behavior of one or more variables associated with the mathematical relation, altering or specifying one or more functions associated with the mathematical relation, or a combination of them.

Each identified biological process is modified across a range scaled from a starting value. In one implementation, the starting value is determined by the computer model for a particular virtual patient using a particular set of characteristics. Alternatively, the user establishes a specified starting level using experimental data (e.g., data collected using biological assays), clinical data, knowledge or opinion of persons skilled in the art, outputs of the computer model, and other relevant sources. The parameters for each identified biological process are modified so that each identified biological process is scaled down from the starting value, for example, by a factor of 100 or scaled up from the starting value, for example, by a factor of 100. The effects of these modified processes are modeled for each biological process.

### Execute Model with Modified Biological Processes (Step 120)

As discussed previously, the computer model includes modeled processes that represent various biological processes of the biological system. At step 120, the modified parameters for each identified biological process are input into the computer model and modeled to examine the effects of the modifications on the clinical outcomes. For example, changes in identified processes associated with rheumatoid arthritis are used to examine the connection between the process and the disease state by observing effects on outputs for synovial cell density and cartilage degradation rate. A baseline output is produced by running the computer model 200 is run in the absence of a modification of the various biological processes. The computer model 200 is also run with the modification of the various biological processes to provide one or more outputs. The unmodified output is compared with one or more modified outputs to identify the degree of connection between one or more biological processes and the clinical outcomes. A high degree of connection can indicate a potential therapeutic target based on the identified biological process.

In one implementation, outputs are compared using a sensitivity analysis. Sensitivity analysis involves prioritization of biological processes that are associated with the disease state. Sensitivity analysis is performed with different configurations of the computer model to determine robustness of the prioritization. In some instances, sensitivity analysis involves a rank ordering of biological processes based on their degree of connection to the disease state. Sensitivity analysis allows a user to determine the importance of a biological process in the context of the disease state. An example of a biological process of greater importance is a biological process that increases the severity of the disease state. Thus, inhibiting this biological process can decrease the severity of the disease state. The importance of a biological process depends not only on the existence of a connection between that biological process and the disease state, but also on the extent to which that biological process has to be modified to achieve a change in the severity of the disease state. In a rank ordering, a biological process playing a more important role in the disease state typically receives a higher rank. The rank ordering can also be done in a reverse manner, such that a biological process that plays a more important role in the disease state receives a lower rank. Typically, the set of biological processes include biological processes that are identified as playing a more important role in the disease state.

For each biological process, the computer model 200 is run using the modification of the modeled biological process to produce a comparison output associated with the biological process. The comparison output is then compared with the baseline output. The computer model 200 is run using all the modifications of the various biological processes to produce a baseline output where all the effects are applied. Next, for each modeled biological process, the computer model is run in the absence of the modification of the modeled biological process to produce a comparison output associated with the biological process. The comparison output is then compared with the baseline output.

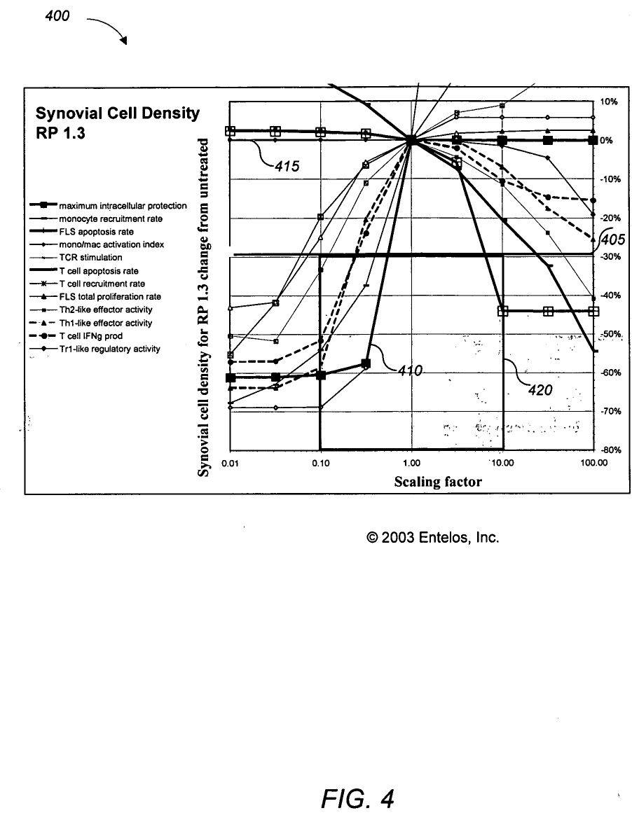

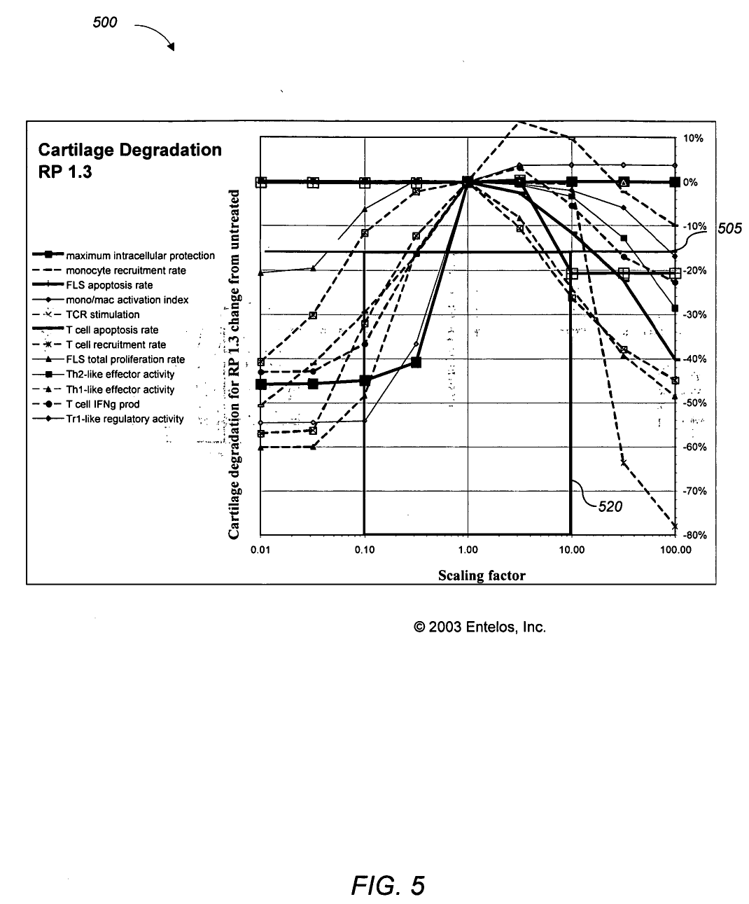

For example, FIGS. 4 and 5 illustrate outputs from the computer model 200 (a portion of which is shown in FIG. 2) that illustrate the effects of modifying each of the identified processes on a virtual patent having rheumatoid arthritis. The computer model 200 introduces modifications to an modeled biological process as different virtual stimuli. The outputs of the virtual patent in response to the virtual stimuli are expressed as changes in clinical outcomes associated with rheumatoid arthritis including synovial cell density and cartilage degradation rate. Therefore, the biological processes, modified to affect synovial cell density and/or cartilage degradation, are potential therapeutic targets for treating rheumatoid arthritis.

FIG. 4 shows a graph 400 of the effects that modifications of different identified processes have on synovial cell density according to the computer model 200. In FIG. 4, a number of identified processes are charted showing the percent change in synovial cell density for a virtual patent (e.g., virtual patient RP 1.3) with rheumatoid arthritis. For each identified process, the parameters are modified to provide a change in the process along a range from a starting value to an increase or decrease by a factor of 100. Each identified process is separately modified while other processes are held constant. Each process is then overlaid on the same graph such that the outputs for each identified process are compared. In another implementation, more than one process is modified simultaneously.

In addition to the identified biological processes, FIG. 4 illustrates the effect of applying methotrexate, the standard treatment, on synovial cell density. Line 405 illustrates the effect of methotrexate on the virtual patient RP 1.3. Accordingly, methotrexate reduces the synovial cell density by 30% from the untreated state. Some biological processes appear to have a greater connection to synovial cell density than other processes. For example, when maximum intracellular protection 410, which controls the rate of macrophage apoptosis, is reduced the synovial cell density is reduced sharply and then levels off at a reduction of substantially 60% from an untreated patient. In contrast, another identified biological process, tr1-like regulatory activity 415, leaves synovial cell density substantially unchanged when reduced or enhanced. Consequently, synovial cell density appears to be more sensitive to particular biological processes than to others.

FIG. 5 shows a graph 500 of the effects of the same modifications to the same modeled biological processes on cartilage degradation rate. Again, the effect of the therapy, methotrexate 505 is shown along with lines charting the output effects of increases or decreases in the identified biological processes. As with synovial cell density shown in FIG. 4, cartilage degradation rate appears to be more sensitive to particular biological processes than to others. Similarly, the effects of the identified biological processes on other clinical outcomes of rheumatoid arthritis (e.g., IL-6 level or rate of bone erosion) can also be modeled.

Identify Potential Targets (Step 125)

Referring back to FIG. 1, using data from the modifications of the identified biological processes, for example, using the graphs in FIGS. 4 and 5, potential therapeutic targets are identified at step 125. Referring back to FIGS. 4 and 5, values for the identified biological processes associated with rheumatoid arthritis were scaled down by a factor of 100 and scaled up a factor of 100. However, in identifying potential therapeutic targets, a user can consider practical limitations on the ability to affect the identified biological process. For example, it may not be possible or safe to increase the functioning of a biological process by a factor of 100. In one implementation, bounds on the ability to affect the biological process are placed at a factor of ten in both reduction and enhancement of the biological process. FIGS. 4 and 5 illustrate boxes 420 and 520 respectively indicating a reasonable bounds of the ability to affect the biological constituents. The boxes 420 and 520 are capped by the performance of methotrexate 405 and 505. Boxes 420 and 520 a region of greatest interest in identifying potential targets. Biological processes falling within the boxes 420 and 520 are within the range most likely amenable to potential practical modification and performing better than methotrexate. Additionally, in one implementation, biological processes falling outside of the boxes 420 and 520 respectively are considered lower priority for further investigation or eliminated from consideration because the biological processes do not appear to sufficiently affect the clinical outcomes (e.g., Tr1-like regulatory activity 415).

For example, in FIG. 4, several of the biological processes fall within box 420. However, by comparing outputs, it is apparent that different biological processes reduce synovial cell density by different degrees. In one implementation, a potential therapeutic target is identified by selecting the biological process having the greatest effect on synovial cell density. In another implementation, a potential therapeutic target is identified by selecting biological process having the greatest effect on synovial cell density with the least amount of modification. Similarly, FIG. 5 illustrates, for cartilage degradation rate, several biological processes falling within box 520. Again, each biological process exhibits varying degrees of effect on cartilage degradation rate for different levels of modification. After identifying important biological pathways, potential molecular targets are identified and the potential targets are examined for use as a target in the treatment of the disease state (e.g., rheumatoid arthritis).

Computer model 200 performs sensitivity analysis for various modeled biological processes. The outputs of the sensitivity analysis are expressed as effects on clinical outcomes, including cartilage degradation rate, synovial cell density, rate of bone erosion, and IL-6 level. The sensitivity analysis is used to identify and compare particular biological processes having a significant effect on the clinical outcomes. In one implementation, sensitivity analysis identifies four areas of the biology of rheumatoid arthritis having a significant effect on the disease pathophysiology: (1) macrophage apoptosis, (2) interferon-gamma production, (3) Th1 cell activation, and (4) T-cell and monocyte recruitment.

### Examine Potential Targets (Step 130)

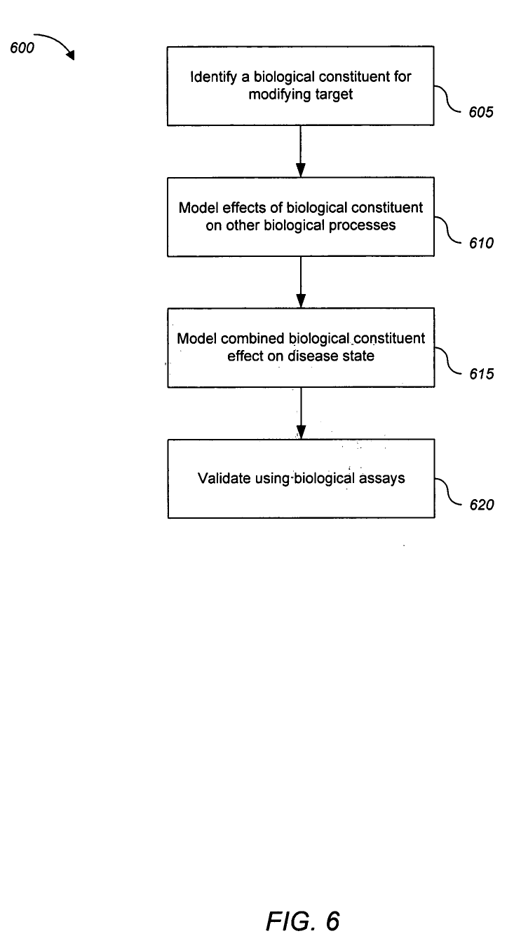

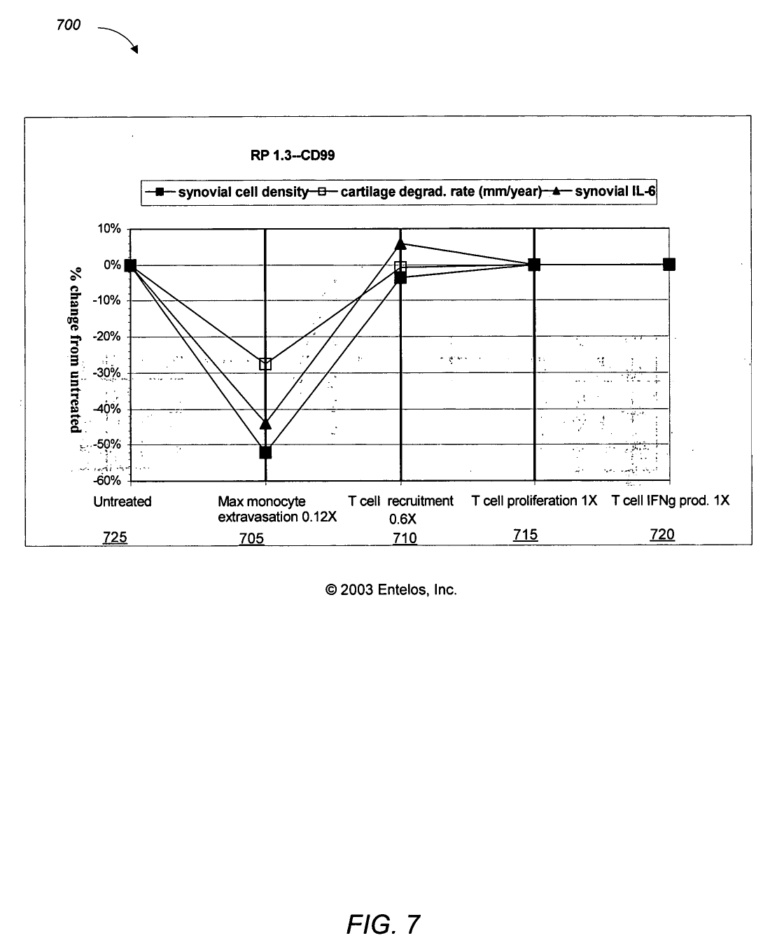

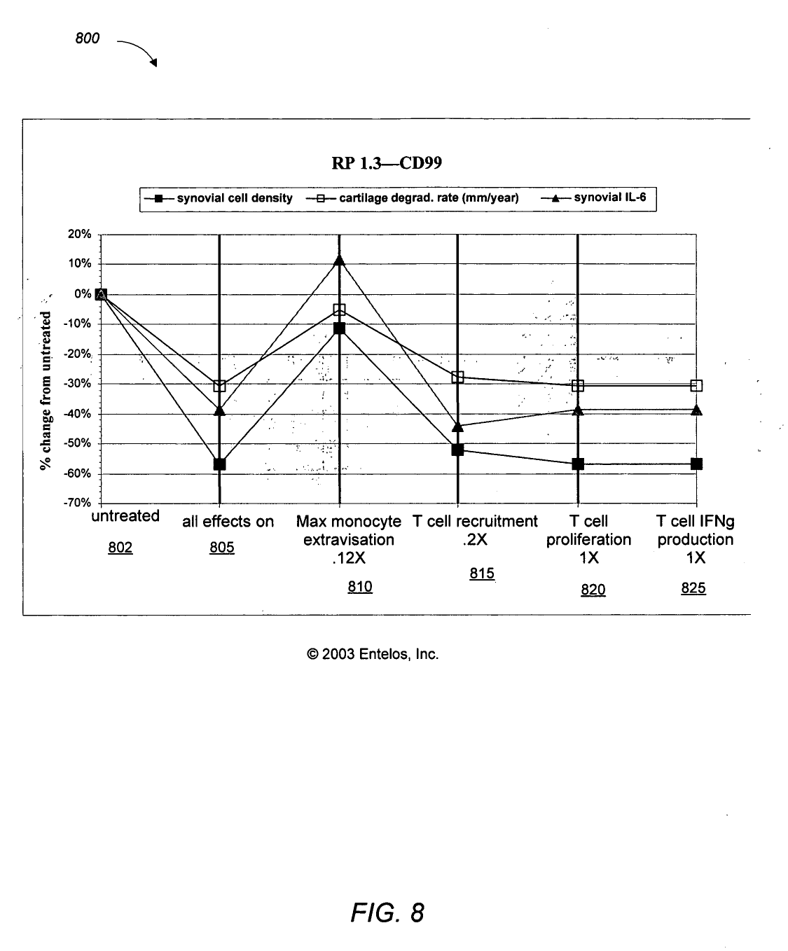

Referring again to FIG. 1, after one or more processes important to the disease state have been identified, each is examined to determine whether modification of the biological process can be used in the treatment of the disease state (step 130). FIG. 6 shows a method 600 for examining potential therapeutic targets. A biological constituent is identified, for example by a user, for modifying the potential target (step 605). Once the biological constituent is identified, the user modifies the computer model 200 to incorporate the biological constituent. The effects of the biological constituent on other biological processes can then be modeled (step 610). The computer model 200 models the biological constituent to show the combined effect of the biological constituent on the clinical outcomes associated with the disease state (step 615). Validation of the modeled effects is performed, for example, using a set of biological assays (step 620). Each step in method 600 is discussed in further detail below.

Identify Biological Constituent

A biological constituent that effects the modification of the potential target is identified at step 605. For example, a user identifies a biological constituent that affects particular functions of the one or more biological processes from FIG. 4 to provide a desired behavior (e.g., a biological constituent that provides a reduction in an identified biological process associated with a value of a clinical outcome shown in box 420 of FIG. 4). A process for identifying a biological constituent capable of performing the desired function to a biological process can include data based on experiments, clinical data, knowledge or opinion of persons skilled in the art, outputs of computer models, and other relevant sources. In one implementation, biological constituent “CD99” is identified as performing the desired effect on a biological process associated with rheumatoid arthritis. In one implementation, CD99 is identified as a biological constituent associated with functions including monocyte extravasation (monocyte recruitment), T-cell recruitment, T-cell proliferation, and T-cell activation. In one implementation, outputs of the computer model predict that CD99 antagonism provides a beneficial therapeutic effect for rheumatoid arthritis.

Include Biological Constituent in Computer Model

Once a biological constituent has been identified (e.g., CD99), the biological constituent is incorporated into the computer model 200 as a model constituent. In one implementation, a set of functions of CD99 associated with monocyte extravasation, T-cell recruitment, T-cell proliferation, and T-cell activation are quantified and incorporated in the computer model 200. Incorporating the functions of CD99 into the computer model 200, allows modeling of the effects on other biological processes associated with rheumatoid arthritis (step 610). FIGS. 7 and 8 show outputs using a sensitivity analysis of CD99. In particular, FIGS. 7 and 8 show graphs 700 and 800 respectively of outputs for a virtual patient (e.g., RP 1.3) representing an arthritic human patient that exhibits appropriate responses to common therapies for rheumatoid arthritis (e.g., methotrexate).

Model Combined Effect of Biological Constituent

The behavior of the virtual patient following the introduction of a virtual stimulus is modeled. Each virtual stimulus provides a specified level of modification of a particular biological process (e.g., introducing CD99 to inhibit a particular biological process by a specified amount). In one implementation, a user specified level of modification is established based on experimental data (e.g., data collected using biological assays), clinical data, knowledge or opinion of persons skilled in the art, outputs of the computer model, and other relevant sources. Specifically, in FIG. 7, the introduction of CD99 reduces maximum monocyte extravasation 705 to 0.12x its untreated value, T-cell recruitment 710 to 0.6x its untreated value, and T-cell IFNg Production 720 to 1x its untreated value. The value of T-cell proliferation 715 is unaffected by CD99.

The computer model 200 is run to determine the effect that the changed levels of each of the virtual stimuli (e.g., maximum monocyte extravasation 705) has on clinical outcomes including synovial cell density and cartilage degradation rate. The computer model 200 is run without any modeled virtual stimuli to provide a baseline untreated output 725. Then the computer model 200 is run to using each virtual stimulus to evaluate the effect of CD99 on synovial cell density, cartilage degradation rate, and synovial IL-6. As shown in FIG. 7, the use of CD99 to reduce max monocyte extravasation to 0.12x the untreated value greatly decreases the clinical outcomes associated with rheumatoid arthritis. However, other biological functions, such as T-cell recruitment 710, have little effect on synovial cell density or cartilage degradation rate even though T-cell recruitment 710 is reduced to 0.6x its standard value by CD99. A result showing only a minor effect can indicate, for example, that the clinical outcomes are not as sensitive to T-cell recruitment as first appeared in the initial modeling of the biological process.

While FIG. 7 illustrates singular effects, FIG. 8 illustrates combined effects as chart 800. As with FIG. 7, the computer model 200 is first run without any virtual stimuli to produce a first baseline untreated clinical outcomes. The computer model 200 is also run based on all virtual stimuli at once to produce a second baseline output 805 (labeled as “all effects on”). The computer model 200 is also then be run in the absence of one virtual stimulus at a time and using all remaining virtual stimuli to produce a comparison output associated with a particular biological process or function (e.g., all stimuli but maximum monocyte extravasation 810, all stimuli but T-cell recruitment 815, all stimuli but T-cell proliferation 820, or all stimuli but T-cell IFNg production 825). Outputs of the virtual stimuli are expressed as a percentage change the clinical outcomes of synovial cell density, cartilage degradation rate, and synovial IL-6 level compared to an untreated condition 802. As shown in FIG. 7 and FIG. 8, the outputs of the virtual stimuli indicate that inhibition of a function associated with monocyte extravasation has a potential for affecting a disease state.

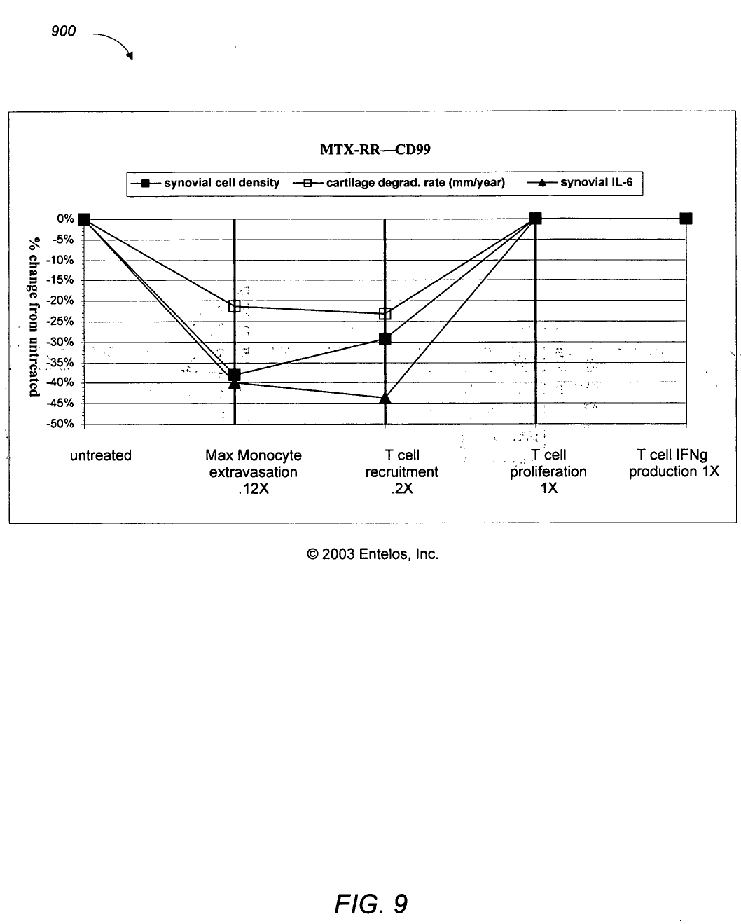

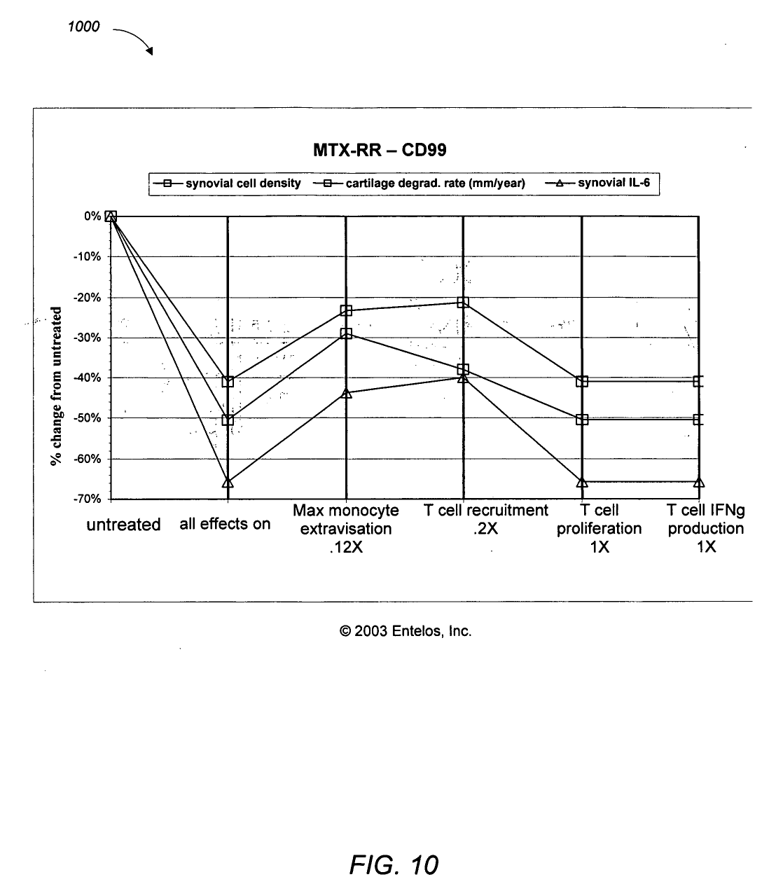

In one implementation, different virtual patients are modeled to evaluate the effect of modified stimuli on virtual patients having different characteristics. FIGS. 9 and 10 show additional outputs based on sensitivity analysis of various modeled biological processes or functions modified by the biological constituent CD99. In particular, FIGS. 9 and 10 show charts 900 and 1000 of outputs for virtual patient MTX-RR, which again represents an arthritic human patient that exhibits reduced response to methrotexate. Various virtual stimuli modeled to evaluate the behavior of the virtual patient based on the virtual stimuli. The clinical outcomes for the virtual stimuli are shown for the virtual patient. Again, each virtual stimulus is implemented to simulate a specified level of modification of a particular biological process or function. As was shown in FIGS. 7 and 8, the user can specify a level of modification using experimental data (e.g., data collected using biological assays), clinical data, knowledge or opinion of persons skilled in the art, outputs of the computer model, and other relevant sources.

As shown in FIG. 9, the computer model 200 is run without any of the virtual stimuli to provide a baseline untreated output representing an untreated state. The computer model 200 is then be run using one modeled virtual stimulus at a time to provide one or more comparison outputs of the clinical outcomes. As shown in FIG. 10, the computer model 200 is run without any of the virtual stimuli to produce a first baseline untreated output. The computer model 200 can then be run using all the virtual stimuli at once to produce a second baseline output (labeled as “all effects on”) and is then run with the reduction of one virtual stimulus at a time to then provide an outcome including all remaining virtual stimuli. The varying outcomes provide comparison outputs for the clinical outcomes. Outputs of the virtual stimuli are again expressed as a percentage change in the clinical outcomes of synovial cell density, cartilage degradation rate, and synovial IL-6 level. As shown in FIG. 9 and FIG. 10, the outputs of the virtual stimuli indicate that inhibition of particular biological processes or functions associated with monocyte extravasation and T-cell recruitment have a potential for affecting rheumatoid arthritis by affecting the clinical outcomes. Particularly, FIGS. 7-10 also illustrate the specific level of inhibition that a CD99 blocker needs to have to be an effective therapy for a standard patient type or a methotrexate resistant patient type.

Various biological processes or functions can be tested in combination using computer model 200 instead of being tested individually. For example, the computer model 200 is run without any modification to first provide a baseline output. Next, a modification is modeled for each biological process or function. The computer model 200 is then run using one or more of the modifications to produce one or more outputs. The outputs are compared with the baseline output. In one implementation, testing of different modifications to biological processes or functions in combination is performed with different configurations of the computer model 200 to determine robustness of the results.

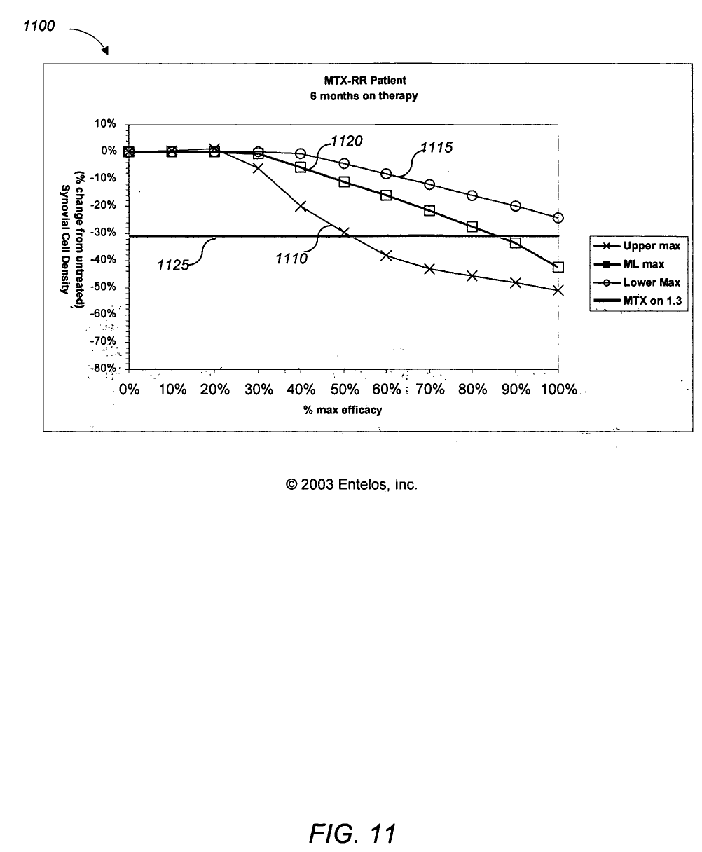

In addition to modeling the effects of the biological constituent on other biological processes or functions associated with a disease state, the combined effect of a biological constituent on clinical outcomes is modeled (step 615). FIG. 11 shows outputs based on testing of various biological processes and functions affected by the biological constituent CD99 in combination. In particular, FIG. 11 shows a chart 1100 of outputs for the virtual patient MTX-RR representing an arthritic human patient that exhibits reduced response to methrotexate. The outputs in FIG. 11 illustrate the effect of CD99 on the clinical outcome of synovial cell density from an untreated state through varying degrees of modeled efficacy. Various virtual stimuli (e.g., different biological processes or functions affected by CD99) are modeled to evaluate the behavior of the virtual patient. The outputs for the virtual stimuli are shown for the virtual patient. Outputs of the virtual stimuli are expressed as a percentage change in synovial cell density.

The computer model 200 can model different levels of effect on synovial cell density, for example, when the role of a biological constituent is not clearly characterized. For example, FIG. 11 shows an upper maximum 1110, lower maximum 1115, and midline 1120 for the effect of CD99 on virtual patient MTX-RR. The effect of methotrexate on virtual patient 1.3 is shown in FIG. 11 as line 1125 illustrating a 30% change in synovial cell density. The computer model 200 is run without any of the virtual stimuli to produce a baseline output 1105 along the y-axis illustrating a 0% maximum efficacy. The computer model 200 is then run based on various virtual stimuli in combination to produce comparison outputs associated with the various biological processes or functions in combination for different levels of efficacy.

The range of effects is defined in order to characterize the contribution of CD99 to the biological processes. Table 1 illustrates the range of effects for some of the biological processes.

TABLE 1

| | Lower | Most likely | Upper |
|---|---|---|---|
| Hypothesis | max effect | max effect | max effect |
| monocyte recruitment | 66% | 88% | 88% |
| T cell proliferation | 0% | 0% | 40% |
| T cell activation | 0% | 0% | 84% |
| T cell recruitment | 20% | 40% | 88% |

The “lower max effect” value represents the lowest observed contribution to a particular biological process, taking in consideration possible redundancies with other proteins; the “upper max effect” represents the maximum observed contribution to a particular biological process; and the “most likely max effect” represents an estimation of a realistic contribution to a particular biological process, taking in consideration the in vivo environment and redundancies.

Outputs of the computer model shown in FIG. 11 illustrate that CD99 antagonism for 6 months can improve the rheumatoid arthritis clinical outcomes by synovial cell density by 40% to 70%. Methotrexate is known to decrease synovial cell density by approximately 30%. At 100% efficacy of inhibition, the computer model predicts that CD99 antagonism can induce a greater improvement than methotrexate. In particular, the computer model predicts that compounds causing 70% inhibition of biological processes or functions associated with CD99 perform better than methotrexate in decreasing synovial cell density. Other clinical outcomes can be similarly modeled, such as cartilage degradation rate, in order to fully asses the effect of CD99 on the disease state.

The comparison of outputs of the computer model can be performed quantitatively or qualitatively. For example, outputs are compared to identify a difference (if any) between the outputs, and the difference is then compared with a threshold value. The threshold value represents a therapeutic efficacy value and is established based on experimental data, clinical data, knowledge or opinion of persons skilled in the art, outputs of the computer model, and other relevant sources. Modified biological processes or functions providing outputs that exceed the threshold value are identified as playing a more important role in the disease state. As another example, outputs are represented graphically (e.g., FIGS. 7-11), and the comparison is performed by the user from visual techniques.

If outputs of the computer model indicate that none of identified biological processes or functions sufficiently affect the disease state, the biological constituent need not be further evaluated as a therapeutic target. However, if the outputs of the computer model indicate that at least one biological process or function sufficiently affects the disease state, then the biological constituent is identified as a therapeutic target.

Validation of Biological Constituent

Validation is performed on the identified biological constituent using a set of biological assays (step 620). In some instances, data collected using the set of biological assays is used to re-evaluate the biological constituent. Biological assays include, for example, cell-based assays and animal models. Cell-based assays are performed with, for example, acute cultures (e.g., cells surgically removed from human or animal tissue and then cultured in a dish) or cell line cultures (e.g., cells that have been transformed to immortalize them). Cells may be derived from normal humans or from humans having a disease. Cells may also be derived from non-human animals such as rats, mice, and so forth. For example, cells may be derived from normal non-human mammals or from non-human mammals that are animal models of a disease. Animal models can include, for example, non-human mammals such as mice, rats, and so forth. The animal models used can include non-human mammals having a disease. For example, animal models of obesity or diabetes can include homozygous obese (ob), diabetic (db), fat (fat), or tubby (tub) mice.

For example, if a particular biological process or function modified by the biological constituent is identified as affecting the disease state, a set of biological assays are identified to validate a connection between the biological process or function and the biological constituent (e.g., validating a connection between macrophage apoptosis and CD99). For example, the biological constituent is modulated in the set of biological assays, and the effect of this modulation is evaluated by measuring the effect on the biological process or function. The results of the sensitivity analysis can be used to prioritize the validation experiments. For example, the effect on macrophage recruitment can be tested in a lab first, and if the outcome is good, a user can proceed with some confidence. If the lab tests on macrophage recruitment are not good, other tests may have positive results, but they are unlikely to cause a beneficial effect on the disease state.

Techniques for measuring levels or activities of biological constituents includes measurements of transcription, translation, and activities of the biological constituents. Measurement of transcription is performed, for example, using a set of probes that include a set of polynucleotide sequences. For example, probes may include DNA sequences, RNA sequences, copolymer sequences of DNA and RNA, sequences of DNA analogs or mimics, sequences of RNA analogs or mimics, or combinations of them. Polynucleotide sequences of probes may be synthesized nucleotide sequences, such as synthetic oligonucleotide sequences. These polynucleotide sequences can be synthesized enzymatically in vivo, enzymatically in vitro (e.g., by polymerase chain reaction), or non-enzymatically in vitro. The set of probes used can be immobilized to a solid support or surface, which may be porous or non-porous. For example, the set of probes may include polynucleotide sequences that are attached to a nitrocellulose or nylon membrane or filter. The set of probes can be implemented as hybridization probes as, for example, disclosed in Sambrook et al., Eds., Molecular Cloning: A Laboratory Manual, Vols. 1-3 (Cold Spring Harbor Laboratory, Cold Spring Harbor, N.Y., 2nd ed. 1989). A solid support or surface may be a glass or plastic surface. In some instances, measurement of transcription can be made by hybridization to microarrays of probes. A microarray typically includes a solid support or surface with an ordered array of binding or hybridization sites for products of various genes (e.g., a majority or substantially all of the genes) of a genome of a biological system. Such microarray can include a population of polynucleotide sequences (e.g., a population of DNA sequences or DNA mimics or a population of RNA sequences or RNA mimics) immobilized to the solid support or surface.

Measurement of translation can be performed according to several methods. For example, whole genome monitoring of proteins using “proteome” techniques can be performed by constructing a microarray in which binding sites include immobilized monoclonal antibodies specific to various proteins encoded by a genome. Antibodies can be present for a substantial fraction of the encoded proteins or at least for those proteins relevant to the action of the therapy being studied. Monoclonal antibodies can be produced as, for example, disclosed in Harlow and Lane, Antibodies: A Laboratory Manual (Cold Spring Harbor, N.Y., 1988). In some instances, monoclonal antibodies can be raised against synthetic peptide fragments, which are designed based on genomic sequence of a cell. For a monoclonal antibody array, proteins from a cell are contacted to the microarray, and binding of the proteins can be assayed with conventional techniques. Alternatively, proteins can be separated by two-dimensional gel electrophoresis systems as, for example, disclosed in Hames et al., Gel Electrophoresis of Proteins: A Practical Approach (IRL Press, New York, 1990); Shevchenko et al., 1996, Proc. Natl. Acad. Scie. U.S.A. 93:1440-1445; Sagliocco et al., 1996, Yeast 12:1519-1533; and Lander, 1996, Science 274:536-539. Two-dimensional gel electrophoresis typically involves iso-electric focusing along a first dimension followed by SDS-PAGE electrophoresis along a second dimension. The resulting electropherograms can be analyzed by numerous techniques, including, for example, mass spectrometric techniques, western blotting, immunoblot analysis using polyclonal and monoclonal antibodies, and internal and N-terminal micro-sequencing. Such techniques allow identification of a substantial fraction of proteins produced under given physiological conditions, including, for example, in cells (e.g., yeast) exposed to a drug or in cells modified by deletion or over-expression of a particular gene.

Measurement of activities of biological constituents, such as proteins, can be performed according to several methods. Measurement of activity can be performed by any functional, biochemical, or physical methods appropriate to the activity being characterized. Where the activity involves a chemical transformation, cellular protein can be contacted with a natural substrate, and the rate of transformation can be measured. Where the activity involves association in multimeric units (e.g., association of an activated DNA binding complex with DNA), the amount of associated protein or secondary consequences of the association (e.g., amounts of mRNA transcribed) can be measured. Also, where a functional activity is known, as in cell cycle control, performance of the functional activity can be measured.

### Alternative Implementations

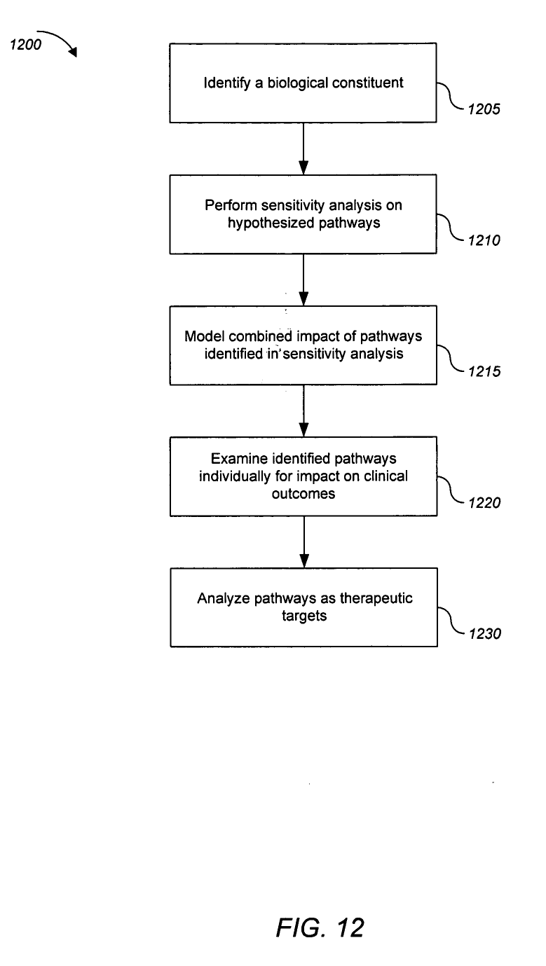

In another implementation, identifying a therapeutic targets begins with a known biological constituent and then identifying biological processes affected by the biological constituent such that the clinical outcomes of interest are effected. FIG. 12 illustrates a method 1200 for structuring an evaluation of a therapeutic target. The biological constituent, such as p38, that is already known to impact a number of functions is identified by a user (step 1205). P38 is present in most cell types and is an important mediator of inflammatory signaling pathways. A user identifies the biological constituent through, for example, a literature search, experimental data, or clinical data. A number of functions associated with p38 are known or hypothesized to impact clinical outcomes based on modifications to p38. User identification of the one or more functions is using, for example, information available in the art regarding the disease state or information available in the art regarding biological processes of the biological system, or a combination of both. For example, a user can identify functions based on experimental data, clinical data, knowledge or opinion of persons skilled in the art, outputs of the computer model, and other relevant sources. In one implementation, more than 100 functions are hypothesized as playing a role in the clinical outcomes for rheumatoid arthritis when p38 is inhibited in those pathways.

A computer model performs a sensitivity analysis to test the hypothesized effect of the biological constituent on each function (step 1205). In one implementation, a computer model already exists for the biological system of interest and includes biological processes influenced by the hypothesized functions. Alternatively, an existing computer model is modified to add biological processes or functions not already incorporated into the model.

In particular, the computer model is run to model a modification of one or more functions of the set of functions. A modification of a function corresponds to an inhibition or a stimulation of a modeled biological process associated with the function, and the modification of the function is represented in the computer model to identify the degree of connection (e.g., the degree of correlation) between the function and the disease state. For example, a modification of a function is modeled to identify the degree that the function affects or is affected by the disease state. For example, the computer model is configured to model the effect the inhibition of p38 for a particular function has on the clinical outcomes such as synovial cell density and cartilage degradation rate.

The sensitivity analysis is performed by the computer model in order to identify which of the hypothesized biological processes or functions actually effect the clinical outcomes when the biological constituent is modified (e.g., inhibition of p38). In one implementation, the sensitivity analysis involves prioritization of functions that are associated with the disease state. This prioritization is used to determine the priority of functions for further scientific investigation and drug characterization. Sensitivity analysis is performed with different configurations of the computer model to determine robustness of the prioritization. In some instances, sensitivity analysis involves a rank ordering of functions based on their degree of connection to the disease state. Sensitivity analysis allows a user to determine the importance of a function in the context of the disease state. The importance of a function depends not only on the existence of a connection between that function and the disease state but also on the extent to which that function has to be modified to achieve a change in the severity of the disease state. In a rank ordering, a function that plays a more important role in the disease state typically receives a higher rank. The rank ordering is also done in a reverse manner, such that a function that plays a more important role in the disease state receives a lower rank.

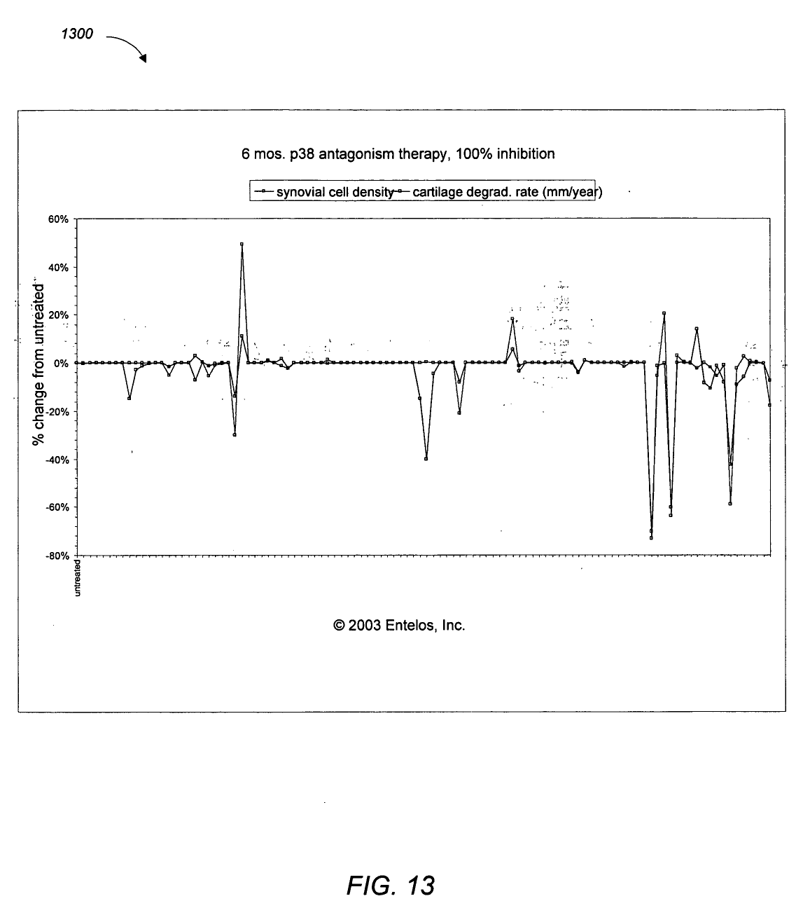

FIG. 13 shows a chart 1300 of outputs based on sensitivity analysis of various potential functions of p38. A virtual patient is defined to represent an arthritic human patient. Various virtual stimuli (e.g., the hypothesized functions) are modeled to evaluate the behavior of the virtual patient based on the virtual stimuli, and outputs associated with the clinical outcomes are shown for the virtual patient based on 100% inhibition of p38. Each virtual stimulus is modeled to simulate a complete inhibition of a particular function, however, other levels of inhibition can also be modeled. The computer model is run based on one virtual stimulus at a time to produce a comparison output for each virtual stimuli with respect to the clinical outcomes. Outputs of the virtual stimuli are expressed as a percentage change in synovial cell density and cartilage degradation rate from an untreated patient. As shown in FIG. 13, the outputs of the virtual stimuli indicate that inhibition of certain functions has a potential for affecting a disease state. In one implementation, sensitivity analysis is performed for additional virtual patients to determine robustness of the results.

The results of the sensitivity analysis are used to reduce the number of functions, or associated biological processes, of interest as potential mechanisms of action of a drug. Consequently, particular functions can be prioritized for further analysis over other functions. As shown in FIG. 13, some modeled biological processes or functions had a greater effect on clinical outcomes than others. Additionally, some results were worse than an untreated state in that, for example, synovial cell density increased instead of decreased. The biological processes or functions indicating the greatest beneficial effect on the clinical outcomes are identified from the results of the sensitivity analysis (step 1215). For example, in one implementation, the sensitivity analysis of p38 reduced the number of functions from over 100 to 16. The 16 remaining functions are then be further analyzed.

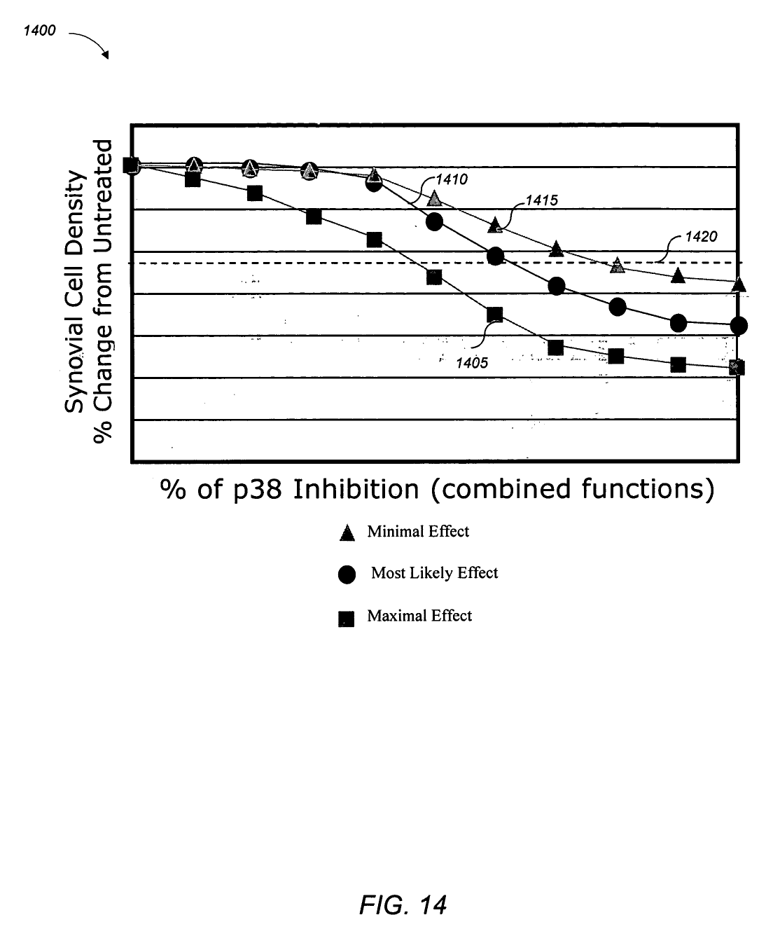

The combined effect of the biological constituent on the clinical outcomes is also modeled (step 1220). The computer model analyzes the combined effect similarly to the techniques shown above with respect to FIGS. 7 and 8. For example, the combined effect on the clinical outcomes of the 16 functions having p38 inhibited are modeled. As shown in FIG. 14, the effect the combined pathways have on the clinical outcomes are modulated based on the degree of p38 inhibition, from zero to 100%, as shown in chart 1400. As before, when the characteristics of p38 are not fully known, predictions of minimal and maximal effects are incorporated into the model. The effect of p38 inhibition is compared for different levels (e.g., maximum 1405, midline 1410, and minimum 1415) as well as for different percentage amounts of inhibition. The effects of p38 are also compared to the effect of methotrexate 1420.

The biological processes or functions are further analyzed by a second level of sensitivity analysis to be narrowed in order to precisely identify pathways important to the clinical benefits of the potential drug. Each individual pathway is individually analyzed for the effect p38 inhibition in that pathway has on the clinical outcomes (step 1225). For example, the 16 functions are individually analyzed. In one implementation, the effects of some biological processes or functions are greater than others. For example, the computer modeled effect of the 16 individual functions results in a determination that only 8 of the 16 are driving the effect of p38 on the clinical outcomes. These 8 functions are then separately analyzed for use as therapeutic targets (step 1230). Thus, the number of potential targets related to a particular known biological constituent is reduced and the set of experiments required for drug evaluation is reduced and prioritized.

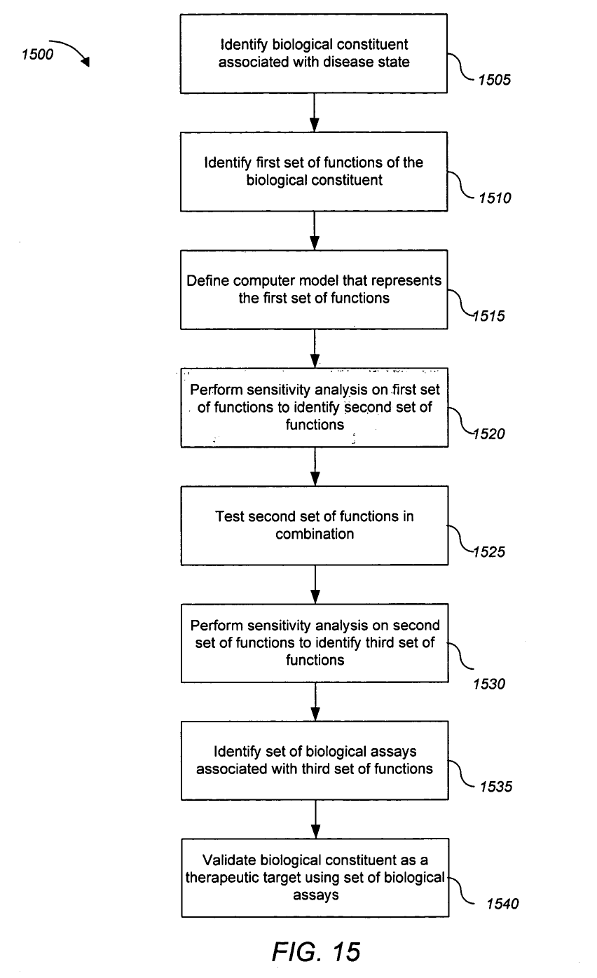

Another example implementation is shown in FIG. 15. FIG. 15 shows a method 1500 for identifying a therapeutic target of a biological system in a disease state. At step 1505, a biological constituent associated with the disease state is identified by a user. The disease state can be associated with, for example, asthma, diabetes, obesity, or rheumatoid arthritis. At step 1510, a first set of functions of the biological constituent is identified. At step 1515, a computer model of the biological system is implemented to represent the first set of functions. Alternatively, previously developed computer model of the biological system is used.

At step 1520, sensitivity analysis is performed on the first set of functions using the computer model. If outputs of the computer model indicate that none of the first set of functions sufficiently affects the disease state, the biological constituent need not be further evaluated as a therapeutic target. However, if outputs of the computer model indicate that at least one function of the first set of functions sufficiently affects the disease state, then the biological constituent is further evaluated as a therapeutic target. Here, sensitivity analysis can identify a second set of functions corresponding to a subset of the first set of functions identified as playing a more important role in the disease state. For certain applications, sensitivity analysis at step 1520 involves simulating complete inhibition of one or more functions of the first set of functions. Also, sensitivity analysis is performed with different configurations of the computer model to determine robustness of the results.

At step 1525, the second set of functions is modeled to determine whether the second set of functions in combination has a potential for affecting the disease state. If outputs of the computer model indicate that the second set of functions in combination does not sufficiently affect the disease state, the biological constituent need not be further evaluated as a therapeutic target. However, if outputs of the computer model indicate that the second set of functions in combination sufficiently affects the disease state, then the biological constituent are further evaluated as a therapeutic target. In some instances, testing the second set of functions at step 1525 is performed with different configurations of the computer model to determine robustness of the results.

At step 1530, sensitivity analysis is performed on the second set of functions using the computer model. If outputs of the computer model indicate that none of the second set of functions sufficiently affect the disease state, the biological constituent need not be further evaluated as a therapeutic target. However, if outputs of the computer model indicate that at least one function of the second set of functions sufficiently affects the disease state, then the biological constituent is identified as a therapeutic target. Here, sensitivity analysis is used to identify a third set of functions corresponding to a subset of the second set of functions that play a more important role in the disease state. For certain applications, sensitivity analysis at step 1530 involves simulating specified levels of modifications of the second set of functions. The modeler can set the specified levels using, for example, experimental data (e.g., data collected using biological assays), clinical data, knowledge or opinion of persons skilled in the art, outputs of the computer model, and other relevant sources. Also, sensitivity analysis is performed with different configurations of the computer model to determine robustness of the results.

At step 1535, a set of biological assays associated with the third set of functions is identified, and, at step 1540, identification of the biological constituent as a therapeutic target is validated based on the set of biological assays. In one implementation, data collected using the set of biological assays is used to re-evaluate the biological constituent in accordance with one or more of the steps shown in FIG. 15.

The invention and all of the functional operations described herein can be implemented in digital electronic circuitry, or in computer hardware, firmware, software, or in combinations of them. The invention can be implemented as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be run on one computer or on multiple computers at one site or distributed across multiple sites and interconnected by a communication network.

Method steps of the invention can be performed by one or more programmable processors executing a computer program to perform functions of the invention by operating on input data and generating output. Method steps can also be performed by, and apparatus of the invention can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

To provide for interaction with a user, the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The invention can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

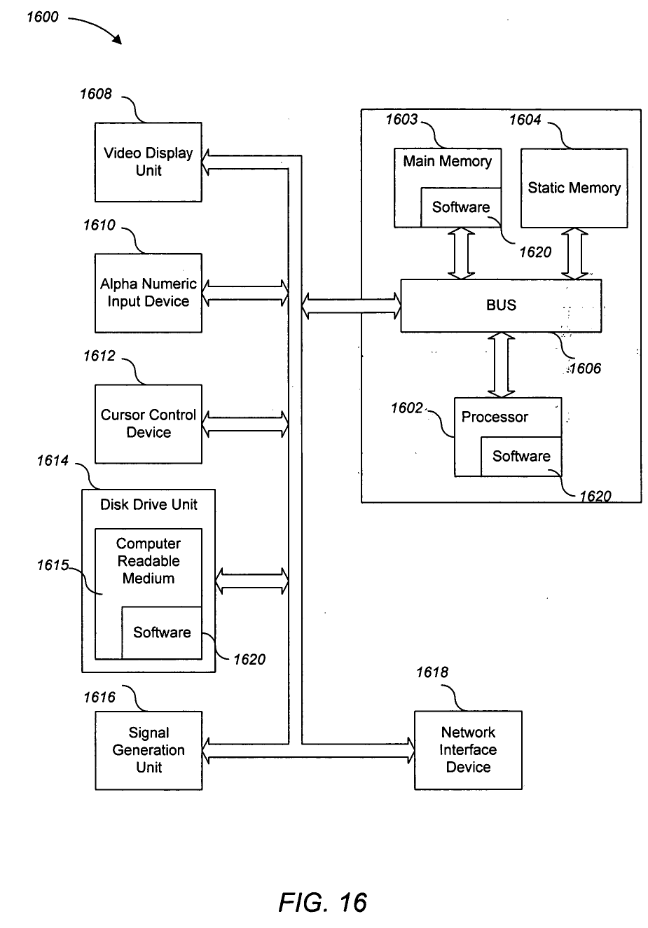

An example of one such type of computer is shown in FIG. 16. FIG. 16 shows a system block diagram of a computer system 1600 that can be operated in accordance with an embodiment of the invention. The computer system 1600 includes a processor 1602, a main memory 1603, and a static memory 1604, which are coupled by bus 1606. The computer system 1600 also includes a video display unit 1608 (e.g., a liquid crystal display (“LCD”) or a cathode ray tube (“CRT”) display) on which a user-interface can be displayed. The computer system 1600 further includes an alpha-numeric input device 1610 (e.g., a keyboard), a cursor control device 1612 (e.g., a mouse), a disk drive unit 1614, a signal generation device 1616 (e.g., a speaker), and a network interface device 1618. The disk drive unit 1614 includes a computer-readable medium 1615 storing software code 1620 that implements processing according to some embodiments of the invention. The software code 1620 can also reside within the main memory 1603, the processor 1602, or both. For certain applications, the software code 1620 can be transmitted or received via the network interface device 1618.

The invention has been described in terms of particular implementations. Other implementations are within the scope of the following claims. For example, the steps of the invention can be performed in a different order and still achieve desirable results.

1. A method of identifying a therapeutic target of a biological system, comprising:
receiving a computer model of a biological system, the model including a plurality of model processes representing a plurality of biological processes and operable to model one or more clinical outcomes associated with a particular disease state;
receiving user input identifying one or more biological processes of the plurality of biological processes, the one or more biological processes being identified as being associated with the one or more clinical outcomes;
modifying, from user input, one or more parameters in the computer model for one or more model processes corresponding to the one or more identified biological processes;
running the computer model using the modified parameters for the one or more model processes to produce output values modeling one or more clinical outcomes; and
identifying one or more modified model processes as a potential therapeutic target.

2. The method of claim 1, wherein identifying one or more model processes includes providing filter information related to the output values.

3. The method of claim 1, further comprising:
providing the output values as a graphical output for the one or more clinical outcomes.

4. The method of claim 1, further comprising:
examining each potential therapeutic target for use as a therapeutic target for treating the disease state, including:
receiving a user identified biological constituent operable to modify a function of a biological process identified as a potential therapeutic target;
receiving user input incorporating a model constituent representing the biological constituent into the computer model of the biological system;
modeling the effect of the model constituent on the one or more model processes associated with the one or more clinical outcomes; and
modeling the effect of the one or more model processes affected by the model constituent on the one or more clinical outcomes.

5. The method of claim 4, further comprising:
validating the effect of the biological constituent on the one or more clinical outcomes using biological assays.

6. The method of claim 1, further comprising:
receiving user input creating the computer model of the biological system.

7. A method of identifying a therapeutic target of a biological system, comprising:
receiving a user identification of a biological constituent selected as a potential therapeutic target for treating a particular disease state;
receiving a computer model of a biological system including a plurality of functions associated and operable to model one or more clinical outcomes associated with a particular disease state;
receiving a user input modifying one or more functions of the plurality of functions affected by the biological constituent;
using the computer model to perform a sensitivity analysis on the one or more functions affected by the biological constituent to identify a set of functions of the one or more functions associated with one or more clinical outcomes; and
modeling the effect of the identified set of functions affected by the biological constituent on the one or more clinical outcomes.

8. A method of identifying a therapeutic target of a biological system in a disease state, comprising:
receiving a user identification of a set of functions of a biological constituent of a biological system;
running the computer model in an absence of a modification of the set of functions to produce a first output;
running the computer model based on the modification of the set of functions to produce a second output; and
comparing the second output with the first output to identify the biological constituent as a therapeutic target.

9. The method of claim 8, wherein the modification of the set of functions comprises modeling an inhibition of at least one function of the set of functions.

10. The method of claim 8, wherein the modification of the set of functions comprises modeling a stimulation of at least one function of the set of functions.

11. The method of claim 8, wherein comparing the second output with the first output includes identifying a difference between the second output and the first output.

12. A method of identifying a therapeutic target of a biological system in a disease state, comprising:
receiving a user identification of a set of functions of a biological constituent of a biological system; and
for each function of the set of functions,
receiving user input defining a modification of the function, the modification of the function corresponding to one of an inhibition of the function and a stimulation of the function;
running a computer model based on the modification of the function to produce a comparison output associated with the function; and
comparing the comparison output with a baseline output.

13. The method of claim 12, wherein the computer model represents a plurality of biological processes of the biological system, and each function of the set of functions is associated with at least one biological process of the plurality of biological processes.

14. The method of claim 13, wherein the computer model represents the plurality of biological processes using a plurality of mathematical relations, and defining the modification of the function includes defining a parametric change in at least one mathematical relation of the plurality of mathematical relations.

15. The method of claim 12, wherein executing the computer model based on the modification of the function includes running the computer model based on the modification of the function and in the absence of the modification of any other function of the set of functions.

16. The method of claim 12, further comprising:
running the computer model in the absence of any of the modifications of the set of functions to produce the baseline output.

17. The method of claim 12, further comprising:
identifying at least one function of the set of functions as having a difference in its associated comparison output with respect to the baseline output.

18. The method of claim 17, further comprising:
receiving user input identifying a set of biological assays associated with the at least one function; and
modifying the at least one function in the set of biological assays to identify the biological constituent as a therapeutic target.

19. A method of identifying a therapeutic target of a biological system in a disease state, comprising:
receiving a user identification of a set of functions of a biological constituent of a biological system;
receiving user input incorporating the set of functions in a computer model of the biological system;
running the computer model in the absence of a modification of the set of functions to produce a first output;
running the computer model based on the modification of the set of functions to produce a second output; and
comparing the second output with the first output to identify the biological constituent as a therapeutic target.

20. The method of claim 19, wherein receiving the user identification of the set of functions includes identifying a set of biological processes of the biological system, the set of biological processes being associated with the set of functions.

21. The method of claim 19, wherein incorporating the set of functions in the computer model includes representing the set of biological processes using a set of mathematical relations.

22. The method of claim 21, wherein executing the computer model based on the modification of the set of functions includes executing the computer model based on a parametric change in at least one mathematical relation of the set of mathematical relations.

23. The method of claim 19, wherein comparing the second output with the first output includes:
identifying a difference between the second output and the first output; and
comparing the difference with a threshold value.

24. A method of identifying a therapeutic target of a biological system in a disease state, comprising:
receiving a user identification of a set of biological processes associated with a biological constituent of a biological system, the set of biological processes being a subset of the plurality of biological processes;
running a computer model in the absence of a modification of the set of biological processes to produce a first output;
running the computer model based on the modification of the set of biological processes to produce a second output; and
identifying a difference between the second output and the first output to identify the biological constituent as a therapeutic target.

25. The method of claim 24, wherein the modification of the set of biological processes corresponds to an inhibition of at least one biological process.

26. The method of claim 24, wherein the modification of the set of biological processes corresponds to a stimulation of at least one biological process.

27. The method of claim 24, wherein the difference between the second output and the first output is predictive of a therapeutic effect of the modification of the set of biological processes on the disease state.

28. A method of identifying a therapeutic target of a biological system in a disease state, comprising:
identifying a biological constituent associated with a disease state;
identifying a set of functions of the biological constituent;
running a computer model in the absence of a modification of the set of functions to produce a first output;
running the computer model based on the modification of the set of functions to produce a second output; and
comparing the second output with the first output to identify the biological constituent as a therapeutic target.

29. The method of claim 28, wherein identifying the biological constituent includes:
identifying a set of biological processes associated with the disease state; and
identifying the biological constituent as being associated with the set of biological processes.

30. The method of claim 29, wherein identifying the set of biological processes includes running the computer model to identify the set of biological processes as contributing to the occurrence of the disease state.

31. The method of claim 30, wherein the computer model represents a plurality of biological processes of the biological system, the set of biological processes is a subset of the plurality of biological processes, and running the computer model to identify the set of biological processes includes:
for each biological process of the plurality of biological processes,
running the computer model based on a modification of the biological process to produce a comparison output associated with the biological process; and
comparing the comparison output with a baseline output.

32. The method of claim 31, wherein executing the computer model to identify the set of biological processes further includes:
identifying the set of biological processes as having differences in their associated comparison outputs with respect to the baseline output.

33. The method of claim 31, wherein the baseline output corresponds to the first output.

34. A method of identifying a therapeutic target of a biological system in a disease state, comprising:
executing a computer model to identify a set of biological processes that contribute to an occurrence of a disease state, the set of biological processes being a subset of a plurality of biological processes;
identifying a biological constituent associated with the set of biological processes;
identifying a set of functions of the biological constituent, each function of the set of functions being associated with at least one biological process of the plurality of biological processes;
running the computer model in the absence of a modification of the set of functions to produce a first output;
running the computer model based on the modification of the set of functions to produce a second output; and
comparing the second output with the first output to identify the biological constituent as a therapeutic target.

35. A computer program product, stored on a computer-readable medium, for identifying a therapeutic target, comprising instructions operable to cause a programmable processor to:
define a computer model of a biological system in a disease state, the computer model representing a set of functions of a biological constituent of the biological system;
define a virtual stimulus, the virtual stimulus representing a modification of the set of functions;
run the computer model in the absence of the virtual stimulus to produce a first output; and
run the computer model based on the virtual stimulus to produce a second output.

36. The product of claim 35, wherein the instructions to define the computer model further comprise instructions to define a plurality of biological processes of the biological system using a plurality of mathematical relations, and each function of the set of functions is associated with at least one biological process of the plurality of biological processes.

37. The product of claim 36, wherein the instructions to define the virtual stimulus further comprise instructions to define the virtual stimulus as a parametric change in at least one mathematical relation of the plurality of mathematical relations.

38. The product of claim 35, further comprising instructions to:
identify a difference between the second output and the first output.

39. A product, stored on a computer-readable medium, for identifying a therapeutic target, comprising instructions operable to cause a programmable processor to:
execute a computer model of a biological system in a disease state to produce a baseline output;
define a first virtual stimulus, the first virtual stimulus representing a modification of a first function of a biological constituent of the biological system; and
run the computer model based on the first virtual stimulus to produce a comparison output associated with the first function.

40. The product of claim 39, further comprising instructions to:
define a second virtual stimulus, the second virtual stimulus representing a modification of a second function of the biological constituent; and
run the computer model based on the second virtual stimulus to produce a comparison output associated with the second function.

41. The product of claim 40, further comprising instructions to:
identify at least one of the first function and the second function as having a difference in its associated comparison output with respect to the baseline output.

42. A computer program product, stored on a computer-readable medium, for identifying a therapeutic target, comprising instructions operable to cause a programmable processor to:
receive a computer model of a biological system, the model including a plurality of model processes representing a plurality of biological processes and operable to model one or more clinical outcomes associated with a particular disease state;
receive user input identifying one or more biological processes of the plurality of biological processes, the one or more biological processes being identified as being associated with the one or more clinical outcomes;
modify, from user input, one or more parameters in the computer model for one or more model processes corresponding to the one or more identified biological processes;
run the computer model using the modified parameters for the one or more model processes to produce output values modeling one or more clinical outcomes; and
identify one or more modified model processes as a potential therapeutic target.

43. The product of claim 42, wherein the instructions to identify one or more model processes includes instructions to provide filter information related to the output values.

44. The product of claim 42, further comprising instructions operable to:
provide the output values as a graphical output for the one or more clinical outcomes.

45. The product of claim 42, further comprising instructions operable to:
examine each potential therapeutic target for use as a therapeutic target for treating the disease state, including instructions to:
receive a user identified biological constituent operable to modify a function of a biological process identified as a potential therapeutic target;
receive user input incorporating a model constituent representing the biological constituent into the computer model of the biological system;
model the effect of the model constituent on the one or more model processes associated with the one or more clinical outcomes; and
model the effect of the one or more model processes affected by the model constituent on the one or more clinical outcomes.

46. The product of claim 41, further comprising instructions operable to:
receive user input creating the computer model of the biological system.

47. A computer program product, stored on a computer-readable medium, for identifying a therapeutic target, comprising instructions operable to cause a programmable processor to:
receive a user identification of a biological constituent selected as a potential therapeutic target for treating a particular disease state;
receive a computer model of a biological system including a plurality of functions associated and operable to model one or more clinical outcomes associated with a particular disease state;
receive a user input modifying one or more functions of the plurality of functions affected by the biological constituent;
perform a sensitivity analysis on the one or more functions affected by the biological constituent to identify a set of functions of the one or more functions associated with one or more clinical outcomes; and
modeling the effect of the identified set of functions affected by the biological constituent on the one or more clinical outcomes.
